(19) United States
(12) Patent Application Publication
(10) Pub. No.: US 2023/0352144 A1
(43) Pub. Date: Nov. 2, 2023

(54) SYSTEMS AND METHODS FOR CLASSIFYING THE STATUS OF A TRANSPLANT

(71) Applicant: CareDx, Inc., Brisbane, CA (US)

(72) Inventors: Hao ZHANG, Pacifica, CA (US); Francois COLLIN, Berkeley, CA (US); Steven STONE, Sandy, UT (US); Kunbin QU, Los Altos, CA (US)

(21) Appl. No.: 18/141,226

(22) Filed: Apr. 28, 2023

Related U.S. Application Data
(60) Provisional application No. 63/336,870, filed on Apr. 29, 2022.

(51) Int. Cl. G16H 20/40; C12Q 1/6883; G16H 50/20

(52) U.S. Cl. CPC G16H 20/40; C12Q 1/6883; G16H 50/20; C12Q 2600/106; C12Q 2600/118; C12Q 2600/156

(57) ABSTRACT

Disclosed herein are systems, kits, and methods for classifying the status of a transplant based on expression levels of a plurality of genes from a biological sample of a transplant recipient. The status of a transplant may be classified based on a predictive rejection classification including, but not limited to, antibody-mediated rejection (ABMR), T-cell mediated rejection (TCMR), mixed ABMR+TCMR, and no rejection. The predictive rejection classification may be assigned based on probability rejection scores, and a probability rejection score may be assigned to each rejection label. In some embodiments, the rejection label having the highest probability rejection score amongst the plurality of rejection labels may be assigned as the predictive rejection classification. Non-limiting rejection labels may include ABMR, TCMR, mixed ABMR+TCMR, and no rejection. The probability rejection score of each rejection label may be generated based on a plurality of sets of weights and expression levels of genes.

### CROSS-REFERENCE TO RELATED APPLICATIONS

This application claims the benefit of U.S. Provisional Application No. 63/336,870, filed Apr. 29, 2022, which is hereby incorporated by reference in its entirety.

### FIELD OF THE DISCLOSURE

This disclosure relates generally to systems and methods for classifying the status of a transplant.

### BACKGROUND OF THE DISCLOSURE

Transplantation of cells, tissues, partial or whole organs are life-saving medical procedures in cases where an individual experiences acute organ failure or suffers from some malignancy. Many organs including, but not limited to, heart, kidney, liver, lung, and pancreas can be successfully transplanted, and one of the most common types of organ transplantations performed nowadays is kidney transplantation.

Upon transplantation of non-self (allogeneic) cells, tissues, or organs (allograft) into a recipient, the transplant recipient's immune system recognizes the allograft to be foreign to the body and activates various mechanisms to reject the allograft. Thus, it is necessary to medically suppress such an immune response to minimize the risk of transplant rejection. After transplantation, the status of the transplant may be monitored by a variety of clinical laboratory diagnostics tests including histopathologic assessment of transplant biopsy tissue. The status may be monitored to guide clinical care and immunosuppressive treatment options. Although a histopathological evaluation (e.g., a biopsy) is the current standard for diagnosis of rejection, improving its diagnostic accuracy for determining and monitoring the status of a transplant, such as an organ transplant, is critical due to the invasive nature of the procedure and the associated risk to the transplant, potential sampling error, and subjective nature of histopathological interpretation.

What is needed are systems and methods for classifying and monitoring the status of a transplant, such as an organ transplant, with improved diagnostic accuracy, as provided by the present disclosure.

### BRIEF SUMMARY OF THE DISCLOSURE

A method for classifying a status of a transplant is disclosed. The method comprises: receiving expression levels of a plurality of genes from a biological sample of a transplant recipient; receiving a plurality of sets of weights for the plurality of genes; generating one or more probability rejection scores of one or more rejection labels based on the plurality of sets of weights and the expression levels; and assigning a predictive rejection classification of the biological sample of the transplant recipient based on the one or more probability rejection scores, wherein the predictive rejection classification classifies the status of the transplant. In some embodiments, at least one of the plurality of genes is associated with one or more of: immune cell activation, organ-specific defense against pathogens, regulation of tissue and cellular processes, or transcription regulation. In some embodiments, the predictive rejection classification classifies the status of the transplant as experiencing antibody-mediated rejection (ABMR), T-cell mediated rejection (TCMR), mixed ABMR+TCMR, or no rejection. In some embodiments, the predictive rejection classification classifies the status of the transplant as experiencing antibody-mediated rejection (ABMR). In some embodiments, the predictive rejection classification classifies the status of the transplant as experiencing T-cell mediated rejection (TCMR). In some embodiments, the predictive rejection classification classifies the status of the transplant as experiencing mixed ABMR+TCMR rejection. In some embodiments, the predictive rejection classification classifies the status of the transplant as experiencing no rejection. In some embodiments, generating one or more probability rejection scores of one or more rejection labels comprises: for each rejection label of a plurality of rejection labels, generating a probability rejection score based on the plurality of sets of weights and the expression levels. In some embodiments, each set of weights comprises a weight for a corresponding rejection label. In some embodiments, assigning a predictive rejection classification of the biological sample of the transplant recipient comprises assigning the rejection label having the highest probability rejection score amongst the plurality of rejection labels as the predictive rejection classification. In some embodiments, the plurality of sets of weights for the plurality of genes is from a machine-learning model trained to: receive a discovery dataset from biological samples of a discovery cohort of transplant recipients, wherein the discovery dataset comprises gene expression levels of a plurality of genes and rejection classifications; analyze the gene expression levels of the discovery dataset for associations with the rejection classifications in the discovery dataset; identify a subset of genes from the plurality of genes of the discovery dataset; and generate the plurality of sets of weights for the subset of genes based on the associations between the gene expression levels of the discovery dataset and the rejection classifications of the discovery dataset, wherein each set of weights is associated with one gene of the subset of genes. In some embodiments, the gene expression levels are analyzed by analyzing nucleic acids from the biological samples of the discovery cohort. In some embodiments, the gene expression levels are analyzed by analyzing RNA from the biological samples of the discovery cohort. In some embodiments, at least some of the rejection classifications of the discovery dataset comprise antibody-mediated rejection (ABMR). In some embodiments, at least some of the rejection classifications of the discovery dataset comprise T-cell mediated rejection (TCMR). In some embodiments, at least some of the rejection classifications of the discovery dataset comprise mixed ABMR+TCMR rejection. In some embodiments, at least some of the rejection classifications of the discovery dataset comprise no rejection. In some embodiments, the expression levels of one or more genes from the plurality of genes of the discovery dataset are normalized relative to gene expression levels of one or more reference genes. In some embodiments, the machine-learning model was validated by: acquiring a validation dataset from biological samples of a validation cohort of transplant recipients, wherein the validation dataset comprises gene expression levels for a plurality of genes and rejection classifications; determining one or more computer-determined predictive rejection classifications from the validation dataset; comparing one or more of the rejection classifications in the validation dataset and the one or more computer-determined predictive rejection classifications; and determining a diagnosis accuracy based on the comparison, wherein the diagnosis accuracy is greater than a predetermined value. In some embodiments, the predetermined value is 60 percent, 70 percent, 80 percent, or 90 percent. In some embodiments, at least one gene of the plurality of genes comprises a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMP3, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1. In some embodiments, the transplant recipient received a transplant comprising one or more of: a kidney transplant, a heart transplant, a lung transplant, a pancreas transplant, a liver transplant, an intestinal transplant, or a vascularized composite allograft transplant. In some embodiments, the transplant recipient received a transplant that is an allograft or a xenograft. In some embodiments, the biological sample is an organ tissue sample. In some embodiments, a step of administering an immunosuppressive treatment.

A kit for classifying the status of a transplant is disclosed. The kit may comprise: one or more probesets specific for one or more genes identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1, reagents, controls, and instructions for use. In some embodiments, the kit further comprises instructions for: receiving expression levels of a plurality of genes from a biological sample of a transplant recipient; receiving a plurality of sets of weights for the plurality of genes; generating one or more probability rejection scores of one or more rejection labels based on the plurality of sets of weights and the expression levels; and assigning a predictive rejection classification of the biological sample of the transplant recipient based on the one or more probability rejection scores, wherein the predictive rejection classification classifies the status of the transplant. In some embodiments, the predictive rejection classification classifies the status of the transplant as experiencing antibody-mediated rejection (ABMR), T-cell mediated rejection (TCMR), mixed ABMR+TCMR, or no rejection. In some embodiments, generating one or more probability rejection scores of one or more rejection labels comprises: for each rejection label of a plurality of rejection labels, generating a probability rejection score based on the plurality of sets of weights and the expression levels. In some embodiments, each set of weights comprises a weight for a corresponding rejection label. In some embodiments, the transplant recipient received a transplant comprising one or more of: a kidney transplant, a heart transplant, a lung transplant, a pancreas transplant, a liver transplant, an intestinal transplant, or a vascularized composite allograft transplant. In some embodiments, the transplant recipient received a transplant that is an allograft or a xenograft. In some embodiments, the biological sample is an organ tissue sample. In some embodiments, assigning a predictive rejection classification of the biological sample of the transplant recipient comprises assigning the rejection label having the highest probability rejection score amongst the plurality of rejection labels as the predictive rejection classification.

A system for classifying a status of a transplant is disclosed. The system may comprise: a scoring unit that: receives expression levels of a plurality of genes from a biological sample of a transplant recipient; receives a plurality of sets of weights for the plurality of genes; generates one or more probability rejection scores of one or more rejection labels based on the plurality of sets of weights and the expression levels; and assigns a predictive rejection classification of the biological sample of the transplant recipient based on the one or more probability rejection scores, wherein the predictive rejection classification classifies the status of the transplant. In some embodiments, at least one of the plurality of genes is associated with one or more of: immune cell activation, organ-specific defense against pathogens, regulation of tissue and cellular processes, or transcription regulation. In some embodiments, the predictive rejection classification classifies the status of the transplant as experiencing antibody-mediated rejection (ABMR), T-cell mediated rejection (TCMR), mixed ABMR+TCMR, or no rejection. In some embodiments, the predictive rejection classification classifies the status of the transplant as experiencing antibody-mediated rejection (ABMR). In some embodiments, the predictive rejection classification classifies the status of the transplant as experiencing T-cell mediated rejection (TCMR). In some embodiments, the predictive rejection classification classifies the status of the transplant as experiencing mixed ABMR+TCMR rejection. In some embodiments, the predictive rejection classification classifies the status of the transplant as experiencing no rejection. In some embodiments, generate one or more probability rejection scores of one or more rejection labels comprises: for each rejection label of a plurality of rejection labels, generate a probability rejection score based on the plurality of sets of weights and the expression levels. In some embodiments, each set of weights comprises a weight for a corresponding rejection label. In some embodiments, assign a predictive rejection classification of the biological sample of the transplant recipient comprises assign the rejection label having the highest probability rejection score amongst the plurality of rejection labels as the predictive rejection classification. In some embodiments, the plurality of sets of weights for the plurality of genes is from a machine-learning model trained to: receive a discovery dataset from biological samples of a discovery cohort of transplant recipients, wherein the discovery dataset comprises gene expression levels of a plurality of genes and rejection classifications; analyze the gene expression levels of the discovery dataset for associations with the rejection classifications in the discovery dataset; identify a subset of genes from the plurality of genes of the discovery dataset; and generate the plurality of sets of weights for the subset of genes based on the associations between the gene expression levels of the discovery dataset and the rejection classifications of the discovery dataset, wherein each set of weights is associated with one gene of the subset of genes. In some embodiments, the gene expression levels are analyzed by analyzing nucleic acids from the biological samples of the discovery cohort. In some embodiments, the gene expression levels are analyzed by analyzing RNA from the biological samples of the discovery cohort. In some embodiments, at least some of the rejection classifications of the discovery dataset comprise antibody-mediated rejection (ABMR). In some embodiments, at least some of the rejection classifications of the discovery dataset comprise T-cell mediated rejection (TCMR). In some embodiments, at least some of the rejection classifications of the discovery dataset comprise mixed ABMR+TCMR rejection. In some embodiments, at least some of the rejection classifications of the discovery dataset comprise no rejection. In some embodiments, the expression levels of one or more genes from the plurality of genes of the discovery dataset are normalized relative to gene expression levels of one or more reference genes. In some embodiments, the machine-learning model was validated by: acquiring a validation dataset from biological samples of a validation cohort of transplant recipients, wherein the validation dataset comprises gene expression levels for a plurality of genes and rejection classifications; determining one or more computer-determined predictive rejection classifications from the validation dataset; comparing one or more of the rejection classifications in the validation dataset and the one or more computer-determined predictive rejection classifications; and determining a diagnosis accuracy based on the comparison, wherein the diagnosis accuracy is greater than a predetermined value. In some embodiments, the predetermined value is 60 percent, 70 percent, 80 percent, or 90 percent. In some embodiments, at least one gene of the plurality of genes comprises a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1. In some embodiments, the transplant recipient received a transplant comprising one or more of: a kidney transplant, a heart transplant, a lung transplant, a pancreas transplant, a liver transplant, an intestinal transplant, or a vascularized composite allograft transplant. In some embodiments, the transplant recipient received a transplant that is an allograft or a xenograft. In some embodiments, the biological sample is an organ tissue sample.

### BRIEF DESCRIPTION OF THE DRAWINGS

FIG. 1 illustrates an example system for classifying the status of a transplant, in a biological sample from a recipient of a transplant according to embodiments of the disclosure.

FIG. 2 illustrates a flowchart of an example method for classifying the status of an organ transplant, according to embodiments of the disclosure.

FIG. 3 illustrates an example system for providing expression levels and a plurality of sets of weights of a plurality of genes, according to embodiments of the disclosure.

FIG. 4 illustrates a flow chart of an example method performed by a machine-learning model, according to embodiments of the disclosure.

FIG. 5 illustrates diagrams of example discovery dataset and validation dataset, according to embodiments of the disclosure.

FIG. 6 illustrates a table of example discovery dataset, according to embodiments of the disclosure.

FIG. 7 illustrates a table of example sets of weights for a subset of genes, according to embodiments of the disclosure.

FIG. 8 illustrates a table of example validation dataset, according to embodiments of the disclosure.

FIGS. 9A and 9B illustrate graphs of example diagnosis accuracies for predictive rejection classifications, according to embodiments of the disclosure.

FIG. 10 illustrates an example device that implements the systems and methods disclosed herein, according to embodiments of the disclosure.

### DETAILED DESCRIPTION

Disclosed herein are systems, kits, and methods for classifying the status of a transplant. The status of a transplant may be classified based on expression levels of a plurality of genes from a biological sample of a transplant recipient. The status of a transplant may be classified based on a predictive rejection classification. Example predictive rejection classifications may include, but not limited to, antibody-mediated rejection (ABMR), T-cell mediated rejection (TCMR), mixed ABMR+TCMR, and no rejection. The predictive rejection classification may be assigned based on probability rejection scores. In some embodiments, a probability rejection score may be assigned to each rejection label. In some embodiments, the rejection label having the highest probability rejection score amongst the plurality of rejection labels may be assigned as the predictive rejection classification. Non-limiting rejection labels may include ABMR, TCMR, mixed ABMR+TCMR, and no rejection. The probability rejection score of each rejection label may be generated based on a plurality of sets of weights and expression levels of genes.

The computer-determined status of a transplant may be provided by way of a medical analysis tool that is readily accessible to a physician or medical expert. The medical analysis tool may display the status of a transplant on, e.g., a user interface, a report printout, etc. The physician or medical expert may use the computer-determined status in addition to, or instead of, the physician's or medical expert's assessment of the status of the transplant. The computer-determined status may be provided to the physician or medical expert as the predictive rejection classification and/or probability rejection score(s) for one or more rejection labels. For example, the medical analysis tool may output ABMR, TCMR, mixed ABMR+TCMR, or no rejection as the predictive rejection classification for a given biological sample of a transplant recipient. As another non-limiting example, the medical analysis tool may output 30% ABMR, 50% TCMR, 15% mixed ABMR+TCMR, and 5% no rejection as the probability rejection scores for the rejection labels for a given biological sample of an transplant recipient. The computer-determined status may be used by the physician or medical expert as a guide for treatment options, monitoring protocols, and/or clinical diagnosis.

By quantifying the status of a transplant or classifying the status based on quantified values (e.g., probability rejection scores), the status of a transplant may be objective, consistent, and reliable. The disclosed computer-implemented method can be used to compare the status of a transplant at one point in time to another point in time. Additionally or alternatively, the status may be used as a guide for deciding treatment options and related timing. A systematic assessment may help to better characterize a transplant recipient's response to therapy and help inform subsequent management and care. The results of the computer-implemented methods may be more reproducible such that variations in results between transplant recipients, or from different measurement times for a given transplant recipient, may be reduced.

The plurality of sets of weights may correspond to the plurality of genes and may be received by a machine-learning model. The machine-learning model may generate the plurality of sets of weights based on a discovery dataset (from biological samples from a discovery cohort of transplant recipients) and rejection classifications. The machine-learning model may analyze the gene expression levels of the discovery dataset for associations with rejection classifications in the discovery dataset. A subset of genes from the plurality of genes of the discovery dataset may be identified. A machine-learning model may generate the plurality of sets of weights for the subset of genes. In some embodiments, the plurality of sets of weights may be based on associations between the gene expression levels of the discovery dataset and the rejection classifications of the discovery dataset. In some embodiments, each set of weights may be associated with one gene of the subset of genes. For example, a first set of weights of 100.0, 0.0, 0.0, and 0.0 for no rejection, ABMR, TCMR, and mixed ABMR+TCMR, respectively, may be associated with the gene KIR_Inhibiting_Subgroup_1.

The following description is presented to enable a person of ordinary skill in the art to make and use the various embodiments. Descriptions of specific devices, techniques, and applications are provided only as examples. These examples are being provided solely to add context and aid in the understanding of the described examples. It will thus be apparent to a person of ordinary skill in the art that the described examples may be practiced without some or all of the specific details. Other applications are possible, such that the following examples should not be taken as limiting. Various modifications in the examples described herein will be readily apparent to those of ordinary skill in the art, and the general principles defined herein may be applied to other examples and applications without departing from the spirit and scope of the various embodiments. The various embodiments are not intended to be limited to the examples described herein and shown, but are to be accorded the scope consistent with the claims.

Various techniques and process flow steps will be described in detail with reference to examples as illustrated in the accompanying drawings. In the following description, numerous specific details are set forth in order to provide a thorough understanding of one or more aspects and/or features described or referenced herein. It will be apparent, however, to a person of ordinary skill in the art, that one or more aspects and/or features described or referenced herein may be practiced without some or all of these specific details. In other instances, well-known process steps and/or structures have not been described in detail in order to not obscure some of the aspects and/or features described or referenced herein.

In the following description of examples, reference is made to the accompanying drawings which form a part hereof, and in which, by way of illustration, specific examples are shown that can be practiced. It is to be understood that other examples can be used, and structural changes can be made without departing from the scope of the disclosed examples.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “sample” or “biological sample,” as used herein, refers to any sample obtained from a transplant recipient including, but not limited to, tissue and/or cells from a biopsy, whole blood, plasma, serum, lymph, peripheral blood mononuclear cells, buccal swabs, saliva, or urine.

The term “transplant” includes solid organ transplants as well as hollow organ transplants, e.g., gastrointestinal transplants, from an allogeneic, i.e., non-self, origin within the same species, or across species from a xenogeneic origin, such as a xenotransplant or xenograft. The term “transplant” also includes cellular transplants such as hematopoietic stem cells, pancreatic islet cells, pluripotent cells, skin tissue, skin cells, immune cells including, but not limited to, NK cells and T cells, from allogeneic or xenogeneic origin. The term “transplant” also includes cellular transplants of autologous, i.e., self, origin, e.g., a transplant comprising autologous cells that originate from the recipient, including autologous cells that were genetically engineered before re-administration into the recipient. The terms “transplant” and “allograft” are used interchangeably herein and, in meaning, include a xenograft. A “transplant” refers to any transplant that is transplanted on its own or in combination with one or more transplants.

The term “solid organ transplant,” as used herein, refers to any transplant of a solid organ including, but not limited to, a kidney transplant, a heart transplant, a lung transplant, a liver transplant, a pancreas transplant, a vascularized composite allograft transplant, or combinations of the above transplants.

The term “gene cluster” or “cluster,” as used herein, refers to a group of two or more genes with a related gene expression pattern, e.g., gene expression levels that have a level or degree of correlation or association.

The term “TCMR,” as used herein, refers to cellular or T-cell mediated (allograft or xenograft) rejection including, but not limited to, acute active cellular or T-cell-mediated rejection, chronic active cellular or T-cell-mediated rejection, and chronic stable cellular or T-cell-mediated rejection.

The term “ABMR,” as used herein, refers to antibody-mediated (allograft or xenograft) rejection including, but not limited to, acute active antibody-mediated rejection, chronic active antibody-mediated rejection, and chronic stable antibody-mediated rejection.

The terms “mixed rejection,” and “mixed ABMR+TCMR” refer to rejection that shows characteristics of both ABMR and TCMR.

The terms “no rejection” and “non-rejection,” as used herein, refer to a state that is characterized by an absence of biopsy-confirmed ABMR, TCMR, and/or mixed ABMR+TCMR, or absence of significant rejection-associated clinical symptoms, e.g., as indicated by elevated serum creatinine levels, decreased estimated glomerular filtration rate, abnormal echocardiogram results or some other clinical concern that would indicate a clinical need for a biopsy. The terms “no rejection” and “non-rejection,” as used herein, may also refer to a state that is characterized by low levels of immune activity indicating a resting, quiescent state of the immune system.

The term “nucleic acid,” as used herein, refers to RNA or DNA that is linear or branched, single or double stranded, or a hybrid thereof. The term also encompasses RNA/DNA hybrids.

The term “gene,” as used herein, refers to a nucleic acid, e.g., DNA or RNA, sequence that comprises coding sequences necessary for the production of RNA or a polypeptide. A polypeptide can be encoded by a full-length coding sequence or by any part thereof.

The term “gene expression,” as used herein, refers to the production of a transcriptional or translational product of a gene, e.g., total RNA, mRNA, a splice variant mRNA, or polypeptide. Unless otherwise apparent from the context, gene expression levels can be measured at the RNA and/or polypeptide level. The measurement of gene expression may provide an indication of the presence of transplant rejection or presence of a likelihood or probability of transplant rejection, characterized by elevated activity of cells of the immune system, or an indication of the absence of transplant rejection or absence of a likelihood or probability of transplant rejection, characterized by a resting state of cells of the immune system demonstrating absence of immune activity or low levels of immune activity. The gene expression measurements, optionally normalized relative to gene expression levels of one or more reference genes, may be used to compute probability rejection scores in accordance with an indication of the presence or absence of a probability of transplant rejection. Such probability rejection scores may be used to predict the likelihood of a clinical outcome, e.g., the likelihood of transplant rejection or the likelihood of “no rejection”, in a transplant recipient. For example, such probability rejection scores would enable a treating physician or medical expert to identify transplant recipients who have a high likelihood of “no rejection” and therefore do not require adjustment, e.g., increase, decrease, change, or initiation, of their immunosuppressive treatment, or have a high likelihood of transplant rejection and therefore would require adjustment of their immunosuppressive treatment. The probability rejection scores may be the basis for assigning a predictive rejection classification to classify the status of a transplant.

The term “machine-readable medium,” as used herein, refers to both a single medium and multiple media (e.g., a centralized or distributed database and/or associated caches and servers) that store one or more sets of instructions, and includes any medium that is capable of storing, encoding, or carrying a set of instructions for execution by a device and that causes a device to perform any method disclosed herein and more. The term “machine-readable medium,” as used herein, includes but is not be limited to solid-state memories, optical and magnetic media, and carrier wave signals.

### Example System for Classifying the Status of a Transplant in a Transplant Recipient Post-Transplantation

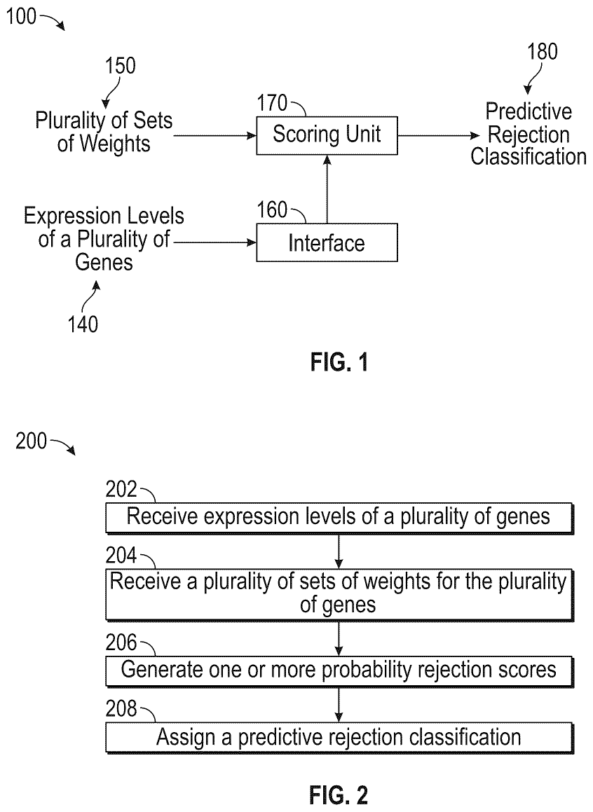

FIG. 1 illustrates an example system 100 for classifying, or determining or assessing, the status of a transplant, for example, an organ transplant, in a biological sample from a recipient of a transplant, according to embodiments of the disclosure. Classifying, determining, and/or monitoring the status of a transplant may be valuable and informative with regard to a clinical decision by a treating physician or medical expert involving the treatment of the transplant recipient, for example, with respect to the need for adjusting, e.g., increasing, decreasing, changing, or initiating, the immunosuppressive or anti-rejection treatment of the transplant recipient.

System 100 may include an interface 160 and a scoring unit 170. Examples of the disclosure may include some or all of the components shown in the figure, or other components not shown in the figure. The system 100 may be, for example, a medical analysis tool. A treating physician or medical expert may use the medical analysis tool to help monitor the status of a transplant in a transplant recipient, as well as monitor and/or suggest an adjustment to an immunosuppressive therapy administered, or to be administered, to a transplant recipient. Monitoring the status of a transplant involves analyzing various aspects that provide useful information about the physiological state of the transplant. The methods of the present disclosure may be used to classify the status of a transplant by way of a predictive rejection classification 180. The predictive rejection classification 180 may indicate the diagnosis that has the highest probability rejection score amongst the plurality of other diagnoses.

The interface 160 may receive the expression levels of a plurality of genes 140 of a biological sample of a transplant recipient. In some embodiments, the expression levels may be provided as user input (e.g., input from a physician or medical expert). The scoring unit 170 may be a tool for assessing the status of a transplant. The scoring unit 170 may receive a plurality of sets of weights 150 (e.g., from a machine-learning model) and the expression levels of a plurality of genes 140. The scoring unit 170 may assign a predictive rejection classification 180 to the biological sample of the transplant recipient.

In some embodiments, system 100 may be a kit used by a treating physician or medical expert for post-transplant monitoring. The kit may classify the status of a transplant, e.g., an organ transplant, in a biological sample from a recipient of a transplant, according to one or more methods disclosed herein. The kit may comprise a set of probesets specific for one or more genes from the plurality of genes.

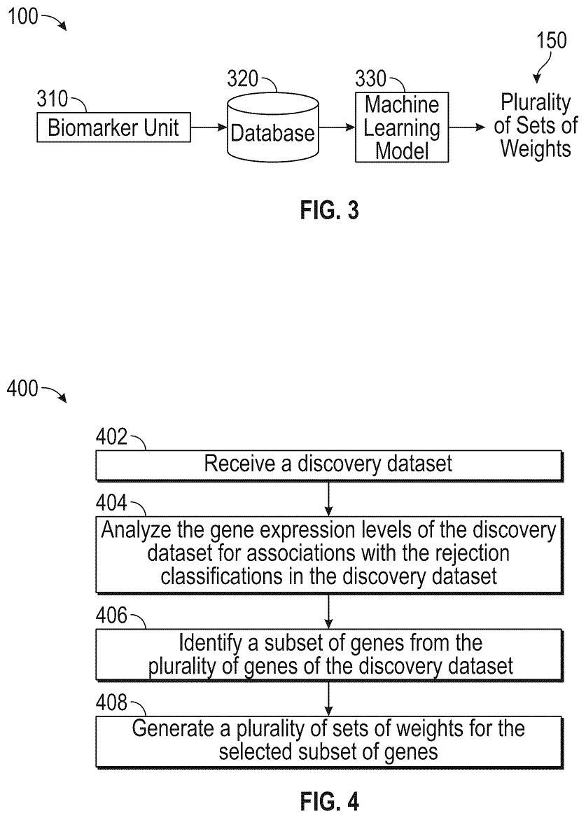

FIG. 2 illustrates a flowchart of an example method for classifying the status of a transplant, e.g., an organ transplant, post-transplantation, according to embodiments of the disclosure. Method 200 may comprise step 202, where the system 100 may receive expression levels of a plurality of genes. The plurality of genes may be from a biological sample of a transplant recipient, e.g., an organ transplant recipient. In some embodiments, the biological sample may be an organ tissue sample. The expression levels are discussed in more detail below.

In step 204, the system may receive a plurality of sets of weights for the plurality of genes. The plurality of sets of weights may be received from a machine-learning model, for example. As discussed in more detail below, the machine-learning model may be trained to generate the plurality of sets of weights based on a discovery dataset and corresponding rejection classifications. As one non-limiting example, each gene, or subset of genes, of the plurality of genes may have a corresponding set of weights. The generation of the plurality of sets of weights is discussed in more detail below.

In step 206, the system may use the scoring unit 170 of FIG. 1 to generate one or more probability rejection scores of one or more rejection labels. The probability rejection score(s) may be based on the plurality of sets of weights (received in step 204) and the expression levels (received in step 202). The probability rejection score for a rejection label may be a percentage value (e.g., between 0% and 100%) indicative of the contribution of the type of rejection to the status of a transplant, e.g. an organ transplant, (in accordance to predictive rejection classification 180 in FIG. 1). A higher probability rejection score may mean a higher contribution. For example, the rejection labels may comprise ABMR, TCMR, mixed ABMR+TCMR, and no rejection. The probability rejection scores for ABMR, TCMR, mixed ABMR+TCMR, and no rejection for a biological sample of, e.g., an organ transplant recipient may be 30%, 50%, 15%, and 5%, respectively. The highest percentage, 50%, may mean that the corresponding rejection label, TCMR, may have the highest contribution to the predictive rejection classification 180 than another rejection label having a lower percentage (e.g., ABMR having a 30% probability rejection score). The generation of the probability rejection scores is discussed in more detail below.

In step 208, the system may assign a predictive rejection classification of the biological sample of a transplant recipient, for example, an organ transplant recipient. The predictive rejection classification may classify the status of the transplant, e.g., the organ transplant. The system may assign each biological sample (e.g., organ tissue sample) one of multiple classifications or diagnoses, such as four diagnoses comprising three different types of rejection and no rejection. A predictive rejection classification may include but is not limited to, ABMR, TCMR, mixed ABMR+TCMR, or no rejection.

In some embodiments, the predictive rejection classification may be assigned based on one or more probability rejection scores. The sum of the probability rejection scores may be equal to 1 or 100%, for example. The rejection label having the highest probability rejection score amongst the plurality of rejection labels may be assigned as the predictive rejection classification, in some embodiments. Returning to the previous example of 30% ABMR, 50% TCMR, 15% mixed ABMR+TCMR, and 5% no rejection for probability rejection scores, the system may assign a predictive rejection classification of TCMR due to TCMR having the highest probability rejection score of 50% amongst the plurality of rejection labels. The assignment of the predictive rejection classification is discussed in more detail below.

Embodiments of the disclosure may include repeating one or more steps of method 200 and/or method 400 (discussed below). Although the descriptions and figures show particular steps of the method occurring in a particular order, the steps of the method may occur in other orders not described or shown. Additionally or alternatively, embodiments of the disclosure may include performing all, some, or none of the steps of method 200 and/or method 400, where appropriate. Furthermore, although certain components, devices, or systems are described as carrying out the steps of method 200 and/or method 400, any suitable combination of components, devices, or systems (including ones not explicitly disclosed) may be used to carry out the steps.

As discussed above, the system 100 (e.g., a medical analysis tool) may receive expression levels of a plurality of genes from a biological sample of a transplant recipient, e.g., an organ transplant recipient. In some embodiments, the expression levels may be used to generate one or more probability rejection scores (e.g., step 206 of method 200 in FIG. 2), where the probability rejection score(s) may be used to assign a predictive rejection classification of the biological sample (e.g., step 208 of method 200 in FIG. 2). The probability rejection score(s) may be based on the expression levels of a plurality of genes with a plurality of sets of weights.

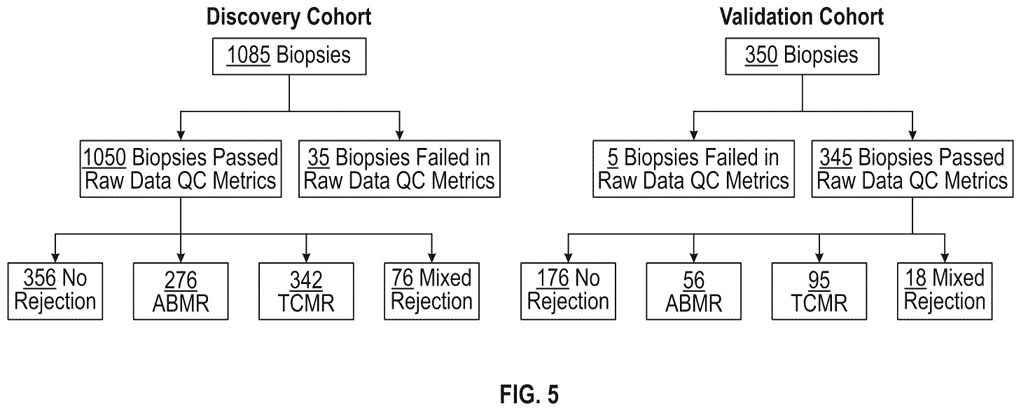

The plurality of sets of weights may be generated by a machine-learning model 330, for example, as shown in the example system of FIG. 3. System 300 may comprise a biomarker unit 310, a database 320, and a machine-learning model 330. The biomarker unit 310 may process and analyze one or more biological samples, including biological samples from a discovery cohort of transplant recipients, e.g., organ transplant recipients. In some embodiments, a biological sample may be an organ tissue sample. The database 320 may store the results from the processing and analysis performed by the biomarker unit 310. The machine-learning model 330 may generate a plurality of sets of weights 150, which may optionally also be stored in the database 320.

In some embodiments, before determining expression levels of a plurality of genes from a biological sample of a transplant recipient, the biological sample may be processed using, e.g., light, immunofluorescence, and electron microscopy. For example, transplant biopsies may undergo immunohistochemical staining for polyomavirus by SV40 on formalin-fixed paraffin embedded (FFPE) tissue or immunofluorescence staining for C4d on unfixed tissue. One or more tissue sections from each FFPE block of renal core biopsy tissue may be dissected using a cutting tool such as a microtome. Dissected tissue sections may be used directly or stored at conditions that maintain the integrity of the nucleic acids, e.g., RNA, and prevent degradation and/or contamination of the tissue sections, until further processed, e.g., for RNA extraction.

The biomarker unit 310 can be configured to determine one or more characteristics of a biological sample of a transplant recipient, e.g., an organ transplant recipient. For example, the biomarker unit 310 may analyze expression levels of a plurality of genes from the biological sample. The transplant recipient may have received a transplant comprising one or more of: a kidney transplant, a heart transplant, a lung transplant, a pancreas transplant, a liver transplant, an intestinal transplant, or a vascularized composite allograft transplant. In some embodiments, the transplant recipient may have received a transplant that is an allograft or a xenograft. In some embodiments, the analysis of the gene expression levels may comprise analyzing for associations with rejection classifications (e.g., of the discovery dataset). In certain embodiments, the analysis of the gene expression levels may comprise analyzing for associations with ABMR. In other embodiments, the analysis of the gene expression levels may comprise analyzing for associations with TCMR. In certain embodiments, the analysis of the gene expression levels may comprise analyzing for associations on the basis of association strength, e.g., low, moderate, or high association strength, as generally interpreted by a person skilled in the art based on the statistical significance of the determined association strength.

Example genes that may be informative with respect to analyzing associations with transplant rejection classifications on the basis of their gene expression levels, and, thus, informative with respect to the status of a transplant in a transplant recipient, in accordance with embodiments of the disclosure, may include, but are not limited to, one or more genes that are associated with immune cell activation, organ-specific defense against pathogens, regulation of tissue and cellular processes, and/or transcription regulation. For example, in some embodiments such genes may be informative, on the basis of their gene expression levels, with respect to whether the transplant recipient experiences “no rejection” or active rejection, e.g., TCMR, ABMR, or mixed ABMR+TCMR. In some embodiments, such genes may be informative, on the basis of their gene expression levels, with respect to the strength of association with one or more rejection classification. The same one or more informative genes may be used for each transplant recipient; there may not be a need to customize the one or more informative genes to different recipients of transplants.

Table 1 lists non-limiting example informative genes that are associated with immune cell activation, organ-specific defense against pathogens, regulation of tissue and cellular processes, and/or transcription regulation; some of these genes belong to correlating gene clusters, as shown in Table 2, exhibiting a correlation of at least 0.6 or 60%. Genes that exhibit a level or degree of correlation of at least 0.6 or 60% with the example informative genes described herein are also considered to be informative, at least on the basis of their gene expression levels, with respect to whether a transplant recipient experiences “no rejection” or active rejection, e.g., TCMR, ABMR, or mixed ABMR+TCMR, and are considered to be within the scope of the present disclosure.

TABLE 1
List of Example Informative Genes

| | | | NCBI NM |
|---|---|---|---|
| | Pathway/ | Pathway/Association | Accession ID or |
| Gene Name | Association | Subgroup | alternative ID |
| KIR_Inhibiting_Subgroup_1 | Immune System | Other Immune Genes | NM_014218.2 |
| GNG11 | Tissue and Cellular Process | Cell Process | NM_004126.3 |
| IL7R | Immune System | Other Immune Genes | NM_002185.3 |
| CXCL8 | Immune System | Chemokine Signaling | NM_000584.2 |
| PLA1A | Tissue and Cellular Process | Metabolism | NM_015900.2 |
| IGHG2 | Immune System | Adaptive Immune System | ENST00000390545.1 |
| KLRK1 | Immune System | Innate Immune System | NM_007360.1 |
| BK large T Ag | Viral Infection | Virus | BKPyVgp5.1 |
| CXCR6 | Immune System | Chemokine Signaling | NM_006564.1 |
| CXCL13 | Immune System | Chemokine Signaling | NM_006419.2 |
| RAPGEF5 | Tissue and Cellular Process | Cell Process | NM_012294.3 |
| FCER1A | Immune System | Innate Immune System | NM_002001.2 |
| DEFB1 | Immune System | Other Immune Genes | NM_005218.3 |
| LGALS3 | Immune System | Other Immune Genes | NM_001177388.1 |
| ROBO4 | Tissue and Cellular Process | Cell Process | NM_019055.5 |
| GNLY | Immune System | Innate Immune System | NM_012483.3 |
| CXCL12 | Immune System | Chemokine Signaling | NM_199168.3 |
| HLA-F | Immune System | Adaptive Immune System | NM_001098479.1 |
| BTG2 | Tissue and Cellular Process | Cell Process | NM_006763.2 |
| SMAD3 | Tissue and Cellular Process | Cell Process | NM_005902.3 |
| CTLA4 | Immune System | Adaptive Immune System | NM_005214.3 |
| HLA-C | Immune System | Adaptive Immune System | NM_002117.4 |
| CASP3 | Tissue and Cellular Process | Apoptosis | NM_004346.3 |
| SH2D1B | Immune System | Adaptive Immune System | NM_053282.5 |
| CXCL11 | Immune System | Chemokine Signaling | NM_005409.4 |
| GBP4 | Tissue and Cellular Process | Cell Process | NM_052941.4 |
| SFTPC | Organ Specific | Lung | NM_001317779.1 |
| SOST | Tissue and Cellular Process | Cell Process | NM_025237.2 |
| PHEX | Tissue and Cellular Process | Cell Process | NM_000444.5 |
| RHOU | Tissue and Cellular Process | Cell Process | NM_021205.5 |
| AGT | Tissue and Cellular Process | Cell Process | NM_000029.3 |
| HSPA12B | Tissue and Cellular Process | Cell Process | NM_052970.4 |
| ENG | Tissue and Cellular Process | Angiogenesis | NM_001114753.1 |
| BMP7 | Tissue and Cellular Process | Cell Process | NM_001719.1 |
| RELA | Tissue and Cellular Process | Cell Process | NM_021975.2 |
| NCAM1 | Immune System | Other Immune Genes | NM_000615.5 |
| NCR1 | Immune System | Other Immune Genes | NM_004829.5 |
| ITGA4 | Tissue and Cellular Process | Cell Process | NM_000885.4 |
| LCN2 | Tissue and Cellular Process | Cell Process | NM_005564.3 |
| HLA-DPB1 | Immune System | Adaptive Immune System | NM_002121.5 |
| COL1A1 | Tissue and Cellular Process | Cell Process | NM_000088.3 |
| XCL1/2 | Immune System | Other Immune Genes | NM_002995.2 |
| BK VP1 | Viral Infection | Virus | BKPyVgp4.1 |
| COL4A1 | Tissue and Cellular Process | Cell Process | NM_001845.4 |
| PLAAT4 | Tissue and Cellular Process | Cell Process | NM_004585.4 |
| ARG2 | Tissue and Cellular Process | Metabolism | NM_001172.3 |
| MCM6 | Tissue and Cellular Process | Cell Process | NM_005915.4 |
| SPRY4 | Tissue and Cellular Process | Cell Process | NM_030964.3 |
| CD81 | Tissue and Cellular Process | Cell Process | NM_004356.3 |
| CD59 | Immune System | Complement System | NM_000611.4 |
| ICAM2 | Immune System | Other Immune Genes | NM_000873.3 |
| RAF1 | Tissue and Cellular Process | Cell Process | NM_002880.3 |
| PLAT | Tissue and Cellular Process | Cell Process | NM_000930.3 |
| CD69 | Immune System | Other Immune Genes | NM_001781.1 |
| CD40LG | Immune System | Adaptive Immune System | NM_000074.2 |
| SMARCA4 | Tissue and Cellular Process | Cell Process | NM_003072.3 |
| NPHS2 | Organ Specific | Kidney | NM_014625.2 |
| IL33 | Immune System | Innate Immune System | NM_001199640.1 |
| CD207 | Tissue and Cellular Process | Cell Process | NM_015717.2 |
| MAPK13 | Tissue and Cellular Process | Cell Process | NM_002754.3 |
| CD58 | Immune System | Other Immune Genes | NM_001779.2 |
| IL1R2 | Immune System | Other Immune Genes | NM_173343.1 |
| TIPARP | Tissue and Cellular Process | Cell Process | NM_015508.3 |
| PSEN1 | Tissue and Cellular Process | Cell Process | NM_000021.2 |
| IGF2R | Tissue and Cellular Process | Cell Process | NM_000876.1 |
| GDF15 | Tissue and Cellular Process | Cell Process | NM_004864.2 |
| AQP2 | Organ Specific | Kidney | NM_000486.5 |
| IL18 | Immune System | Inflammatory Response | NM_001562.3 |
| TNC | Tissue and Cellular Process | Cell Process | NM_002160.3 |
| PECAM1 | Immune System | Other Immune Genes | NM_000442.3 |
| C5 | Immune System | Complement System | NM_001735.2 |
| MICA | Immune System | Other Immune Genes | NM_000247.2 |
| MMP9 | Tissue and Cellular Process | Cell Process | NM_004994.2 |
| EOMES | Immune System | Other Immune Genes | NM_005442.2 |
| EPO | Tissue and Cellular Process | Hematopoiesis | NM_000799.2 |
| EGFR | Tissue and Cellular Process | Cell Process | NM_201282.1 |
| CD2 | Immune System | Other Immune Genes | NM_001767.3 |
| CMV UL83 | Viral Infection | Virus | HHV5wtgp077.1 |
| LYVE1 | Tissue and Cellular Process | Cell Process | NM_006691.3 |
| CD80 | Immune System | Other Immune Genes | NM_005191.3 |
| SIGIRR | Immune System | Other Immune Genes | NM_021805.2 |
| KIT | Tissue and Cellular Process | Cell Process | NM_000222.2 |
| KAAG1 | Organ Specific | Kidney | NM_181337.3 |
| CCL18 | Immune System | Inflammatory Response | NM_002988.2 |
| KLRF1 | Immune System | Other Immune Genes | NM_016523.2 |
| EHD3 | Tissue and Cellular Process | Cell Process | NM_014600.2 |
| BMP2 | Tissue and Cellular Process | Cell Process | NM_001200.2 |
| IL1RL1 | Immune System | Inflammatory Response | NM_016232.4 |
| CD160 | Immune System | Other Immune Genes | NM_007053.3 |
| NOS3 | Tissue and Cellular Process | Cell Process | NM_001160110.1 |
| SERPINE1 | Tissue and Cellular Process | Cell Process | NM_000602.2 |
| CTNNB1 | Tissue and Cellular Process | Cell Process | NM_001098210.1 |
| RASSF9 | Tissue and Cellular Process | Cell Process | NM_005447.3 |
| TFRC | Tissue and Cellular Process | Hematopoiesis | NM_001128148.1 |
| FOXP3 | Tissue and Cellular Process | Cell Process | NM_014009.3 |
| MYB | Tissue and Cellular Process | Hematopoiesis | NM_005375.2 |
| CRHBP | Tissue and Cellular Process | Metabolism | NM_001882.3 |
| CCR7 | Immune System | Adaptive Immune System | NM_001838.3 |
| MT2A | Tissue and Cellular Process | Cell Process | NM_005953.3 |
| CRIP2 | Tissue and Cellular Process | Cell Process | NM_001270837.1 |
| TNFSF9 | Immune System | Other Immune Genes | NM_003811.3 |
| EEF1A1 | Tissue and Cellular Process | Cell Process | NM_001402.5 |
| HLA-B | Immune System | Adaptive Immune System | NM_005514.6 |
| BCL2 | Tissue and Cellular Process | Apoptosis | NM_000657.2 |
| KLF2 | Tissue and Cellular Process | Cell Process | NM_016270.2 |
| CDH5 | Tissue and Cellular Process | Cell Process | NM_001795.3 |
| CD8B | Immune System | Adaptive Immune System | NM_172099.2 |
| SOD2 | Tissue and cellular process | Cell Process | NM_000636.2 |
| SFTPB | Organ Specific | Lung | NM_000542.3 |
| PRDM1 | Tissue and Cellular Process | Cell Process | NM_182907.1 |
| HLA-DQA1 | Immune System | Adaptive Immune System | NM_002122.3 |
| SLC19A3 | Tissue and Cellular Process | Cell Process | NM_025243.3 |
| IFI6 | Tissue and Cellular Process | Apoptosis | NM_002038.3 |
| SIRPG | Tissue and Cellular Process | Cell Process | NM_001039508.1 |
| KLF4 | Tissue and Cellular Process | Cell Process | NM_004235.4 |
| HFE | Immune System | Other Immune Genes | NM_139011.2 |
| MAPK12 | Tissue and Cellular Process | Cell Process | NM_002969.3 |
| SLC4A1 | Tissue and Cellular Process | Cell Process | NM_000342.3 |
| ABCA1 | Tissue and Cellular Process | Cell Process | NM_005502.3 |
| ADORA2A | Tissue and Cellular Process | Cell Process | NM_000675.3 |
| IFIT1 | Tissue and Cellular Process | Cell Process | NM_001548.3 |
| VMP1 | Tissue and Cellular Process | Cell Process | NM_030938.3 |
| JUN | Viral Infection | Viral Detection Genes | NM_002228.3 |
| COL4A4 | Tissue and Cellular Process | Cell Process | NM_000092.4 |
| P2RX4 | Tissue and Cellular Process | Cell Process | NM_001256796.1 |
| SERINC5 | Immune System | Other Immune Genes | NM_001174071.2 |
| BCL2L1 | Tissue and Cellular Process | Apoptosis | NM_138578.1 |
| FABP1 | Organ Specific | Liver | NM_001443.1 |
| TRAF4 | Immune System | Inflammatory Response | NM_004295.2 |
| CCL21 | Immune System | Other Immune Genes | NM_002989.2 |
| RAMP3 | Tissue and Cellular Process | Cell Process | NM_005856.2 |
| PIN1 | Tissue and Cellular Process | Cell Process | NM_006221.2 |
| LOX | Tissue and Cellular Process | Cell Process | NM_002317.4 |
| MAPK3 | Tissue and Cellular Process | Cell Process | NM_001040056.1 |
| CCL3/L1 | Immune System | Inflammatory Response | NM_002983.2 |
| SOX7 | Tissue and Cellular Process | Cell Process | NM_031439.3 |
| CFB | Immune System | Complement System | NM_001710.5 |
| CFH | Immune System | Complement System | NM_000186.3 |
| SFTPD | Organ Specific | Lung | NM_003019.4 |
| HPRT1 | Tissue and Cellular Process | Cell Process | NM_000194.3 |
| TFF3 | Tissue and Cellular Process | Cell Process | NM_003226.3 |
| THBS1 | Tissue and Cellular Process | Cell Process | NM_003246.2 |
| TNFSF4 | Immune System | Adaptive Immune System | NM_003326.2 |
| MYBL1 | Tissue and Cellular Process | Cell Process | NM_001080416.3 |
| CD55 | Immune System | Complement System | NM_000574.3 |
| AGR3 | Tissue and Cellular Process | Cell Process | NM_176813.3 |
| PDPN | Immune System | Other Immune Genes | NM_006474.4 |
| AIRE | Immune System | Adaptive Immune System | NM_000383.3 |
| IL17RB | Immune System | Inflammatory Response | NM_018725.3 |

TABLE 2
List of Genes Correlated with the Example Informative Genes of Table 1

| Name of | Degree of | Degree of | Degree of | Degree of | Degree of | Degree of |
|---|---|---|---|---|---|---|
| Informative | Correlation | Correlation | Correlation | Correlation | Correlation | Correlation |
| Gene | (in brackets) | (in brackets) | (in brackets) | (in brackets) | (in brackets) | (in brackets) |
| GNG11 | TM4SF1 | ERG | ADGRL4 | TM4SF18 | RASIP1 | ECSCR |
| | (0.8) | (0.72) | (0.72) | (0.7) | (0.67) | (0.66) |
| IL7R | IKZF1 | CD96 | ZAP70 | IL16 | CD48 | SLAMF6 |
| | (0.86) | (0.85) | (0.84) | (0.84) | (0.84) | (0.83) |
| CXCL8 | CXCL1/2 | CXCL2 | IL1B | S100A9 | NFKBIZ | CCL20 |
| | (0.79) | (0.73) | (0.7) | (0.65) | (0.65) | (0.64) |
| PLA1A | WARS | IDO1 | APOL1 | GBP1 | CXCL9 | CXCL10 |
| | (0.75) | (0.69) | (0.67) | (0.66) | (0.66) | (0.66) |
| IGHG2 | IGHG3 | IGHG4 | IGHG1 | IGKC | IGLC1 | TNFRSF17 |
| | (1) | (1) | (0.99) | (0.98) | (0.96) | (0.89) |
| KLRK1 | CD8A | CCL5 | IL2RB | GZMA | SLAMF6 | ZAP70 |
| | (0.91) | (0.9) | (0.89) | (0.88) | (0.88) | (0.87) |
| CXCR6 | GZMA | CCR5 | SLAMF6 | CD96 | CD3E | CD3D |
| | (0.89) | (0.88) | (0.87) | (0.87) | (0.87) | (0.86) |
| CXCL13 | ADAMDEC1 | CD84 | PTPN7 | SLAMF6 | SLA | SP140 |
| | (0.79) | (0.78) | (0.78) | (0.78) | (0.77) | (0.77) |
| DEFB1 | HYAL1 | VEGFA | SLC12A3 | UMOD | HDAC6 | RGN |
| | (0.7) | (0.69) | (0.68) | (0.68) | (0.66) | (0.66) |
| ROBO4 | ECSCR | ADGRL4 | RASIP1 | MMRN2 | CD34 | HYAL2 |
| | (0.7) | (0.69) | (0.68) | (0.68) | (0.64) | (0.57) |
| GNLY | NKG7 | PRF1 | CXCL9 | IDO1 | TRDC | TBX21 |
| | (0.76) | (0.76) | (0.76) | (0.74) | (0.73) | (0.72) |
| HLA-F | HLA-E | NLRC5 | PSMB9 | CD74 | IL2RB | TAP1 |
| | (0.95) | (0.94) | (0.94) | (0.94) | (0.93) | (0.92) |
| BTG2 | XBP1 | POU2AF1 | IRF4 | FAM30A | CD79A | TNFRSF17 |
| | (0.72) | (0.69) | (0.66) | (0.65) | (0.65) | (0.65) |
| CTLA4 | SLAMF6 | PTPN7 | SP140 | CD3D | IKZF1 | LCK |
| | (0.89) | (0.89) | (0.89) | (0.88) | (0.88) | (0.88) |
| HLA-C | HLA-E | TAP1 | PSMB9 | HLA-DRB3 | CD74 | TAP2 |
| | (0.79) | (0.78) | (0.78) | (0.77) | (0.77) | (0.76) |
| CASP3 | CASP4 | LY96 | IFNAR2 | IL17RA | ST8SIA4 | BTK |
| | (0.8) | (0.79) | (0.78) | (0.78) | (0.78) | (0.76) |
| SH2D1B | TRDC | NCR1 | TBX21 | PRF1 | KLRC1 | CD45RA |
| | (0.66) | (0.65) | (0.64) | (0.64) | (0.63) | (0.63) |
| CXCL11 | CXCL10 | GBP1 | CXCL9 | IDO1 | WARS | TAP1 |
| | (0.93) | (0.92) | (0.92) | (0.91) | (0.91) | (0.87) |
| GBP4 | GBP1 | WARS | IDO1 | CXCL10 | PSMB9 | CXCL9 |
| | (0.93) | (0.91) | (0.89) | (0.88) | (0.88) | (0.87) |
| PHEX | LAG3 | PTPN7 | ICOS | TIGIT | BTLA | CD72 |
| | (0.72) | (0.7) | (0.69) | (0.69) | (0.69) | (0.66) |
| AGT | CCL15 | LRP2 | ABCC2 | CHCHD10 | RGN | HYAL1 |
| | (0.7) | (0.68) | (0.65) | (0.64) | (0.64) | (0.62) |
| HSPA12B | MMRN2 | CD34 | RASIP1 | ADGRL4 | ECSCR | PALMD |
| | (0.77) | (0.72) | (0.69) | (0.68) | (0.66) | (0.62) |
| ENG | TM4SF1 | HEG1 | PDGFRB | ERG | RASIP1 | BMP4 |
| | (0.69) | (0.67) | (0.64) | (0.64) | (0.59) | (0.59) |
| ITGA4 | PTPRC | CD48 | ZAP70 | IKZF1 | CD247 | INPP5D |
| | (0.72) | (0.72) | (0.71) | (0.71) | (0.71) | (0.71) |
| LCN2 | SERPINA3 | LTF | SLPI | CXCL1/2 | S100A9 | TIMP1 |
| | (0.84) | (0.83) | (0.83) | (0.77) | (0.76) | (0.76) |
| HLA-DPB1 | HLA-DPA1 | CD74 | HLA-DRA | HLA-DMA | HLA-DRB3 | CIITA |
| | (0.97) | (0.96) | (0.95) | (0.94) | (0.94) | (0.93) |
| COL1A1 | COL3A1 | FN1 | CD276 | MMP14 | VCAN | IGF1 |
| | (0.92) | (0.73) | (0.66) | (0.65) | (0.62) | (0.6) |
| XCL1/2 | IL2RB | NLRC5 | CCL5 | PSMB9 | IL2RG | HLA-DPA1 |
| | (0.82) | (0.8) | (0.8) | (0.79) | (0.79) | (0.79) |
| COL4A1 | TIMP1 | VCAN | IFITM2 | IFITM3 | SERPINA3 | NNMT |
| | (0.75) | (0.7) | (0.68) | (0.68) | (0.67) | (0.67) |
| PLAAT4 | PSME2 | LAP3 | PSMB8 | TAP1 | GBP1 | APOL2 |
| | (0.88) | (0.87) | (0.87) | (0.86) | (0.85) | (0.85) |
| MCM6 | DNMT1 | ARHGDIB | CASP4 | CGAS | JAK3 | LY96 |
| | (0.72) | (0.72) | (0.71) | (0.69) | (0.69) | (0.69) |
| PLAT | TEK | TM4SF1 | ERG | HYAL2 | RGS5 | ADGRL4 |
| | (0.7) | (0.56) | (0.56) | (0.54) | (0.5) | (0.5) |
| CD69 | IKZF1 | SLAMF6 | CD96 | CD3E | ZAP70 | CD3D |
| | (0.88) | (0.88) | (0.87) | (0.87) | (0.86) | (0.86) |
| CD40LG | KLRB1 | LTB | TRAT1 | THEMIS | CD96 | IL16 |
| | (0.83) | (0.83) | (0.82) | (0.81) | (0.81) | (0.8) |
| NPHS2 | NPHS1 | PTPRO | VEGFA | MME | | |
| | (0.81) | (0.77) | (0.59) | (0.56) | | |
| AQP2 | UMOD | BMPR1B | GATA3 | COL4A5 | | |
| | (0.74) | (0.73) | (0.67) | (0.6) | | |
| IL18 | C3 | IFNGR1 | S100A9 | CR1 | LILRB4 | FPR1 |
| | (0.66) | (0.65) | (0.64) | (0.64) | (0.64) | (0.63) |
| PECAM1 | ADGRL4 | ECSCR | ACKR1 | EMP3 | LHX6 | MS4A7 |
| | (0.7) | (0.69) | (0.67) | (0.63) | (0.63) | (0.62) |
| CD2 | CD3D | CD3E | CD96 | LCK | ZAP70 | PTPN7 |
| | (0.94) | (0.94) | (0.94) | (0.93) | (0.92) | (0.92) |
| CD80 | NLRC5 | LILRB2 | GBP5 | CIITA | AIM2 | CD48 |
| | (0.68) | (0.67) | (0.67) | (0.67) | (0.67) | (0.67) |
| SIGIRR | HDAC6 | MME | RGN | ALDH3A2 | HNF1A | MAF |
| | (0.73) | (0.69) | (0.67) | (0.67) | (0.65) | (0.65) |
| CCL18 | LILRB4 | IL2RA | CR1 | CD44 | CD84 | LAIR1 |
| | (0.75) | (0.73) | (0.72) | (0.72) | (0.72) | (0.71) |
| CD160 | PIK3CG | TBX21 | NLRC5 | CARD16 | KLRD1 | PRF1 |
| | (0.72) | (0.72) | (0.71) | (0.7) | (0.7) | (0.7) |
| NOS3 | ADGRL4 | ECSCR | HYAL2 | RASIP1 | MMRN2 | ACVRL1 |
| | (0.75) | (0.74) | (0.73) | (0.71) | (0.67) | (0.66) |
| SERPINE1 | CD163 | PTX3 | CDKN1A | MYC | C3AR1 | MRC1 |
| | (0.71) | (0.68) | (0.66) | (0.65) | (0.64) | (0.63) |
| CTNNB1 | IMPDH2 | PDGFRB | CD46 | ACVR1 | | |
| | (0.66) | (0.66) | (0.66) | (0.6) | | |
| RASSF9 | ERG | TM4SF1 | BMP4 | TEK | RGS5 | MMRN2 |
| | (0.7) | (0.69) | (0.67) | (0.63) | (0.62) | (0.6) |
| FOXP3 | TIGIT | IRF4 | SP140 | CXCR5 | FAM30A | CD28 |
| | (0.65) | (0.65) | (0.64) | (0.64) | (0.63) | (0.63) |
| MYB | PTPN7 | JAK3 | BTLA | CD84 | ARHGDIB | PTPN6 |
| | (0.69) | (0.67) | (0.66) | (0.66) | (0.65) | (0.65) |
| CRHBP | MME | VEGFA | NPHS1 | KDR | | |
| | (0.64) | (0.63) | (0.62) | (0.62) | | |
| CCR7 | TLR9 | CD3G | FAM30A | CD3E | IRF4 | POU2AF1 |
| | (0.62) | (0.6) | (0.6) | (0.6) | (0.6) | (0.59) |
| CRIP2 | MMRN2 | NPDC1 | SKI | PDGFA | MCAM | RHOJ |
| | (0.69) | (0.68) | (0.67) | (0.62) | (0.62) | (0.62) |
| EEF1A1 | RPS6 | RPL19 | | | | |
| | (0.83) | (0.68) | | | | |
| HLA-B | HLA-E | PSMB9 | CD74 | TAP1 | NLRC5 | GBP5 |
| | (0.95) | (0.95) | (0.94) | (0.93) | (0.92) | (0.92) |
| CDH5 | ADGRL4 | ECSCR | RASIP1 | MMRN2 | CD34 | CAV1 |
| | (0.8) | (0.76) | (0.75) | (0.7) | (0.68) | (0.66) |
| CD8B | CD8A | TIGIT | CCL5 | SLAMF6 | LCK | LAG3 |
| | (0.82) | (0.78) | (0.77) | (0.76) | (0.75) | (0.75) |
| SOD2 | SERPINA3 | S100A9 | S100A8 | CXCL2 | FPR1 | ADAMTS1 |
| | (0.75) | (0.75) | (0.73) | (0.73) | (0.73) | (0.72) |
| PRDM1 | IRF4 | POU2AF1 | ISG20 | TNFRSF17 | FAM30A | SP140 |
| | (0.89) | (0.86) | (0.84) | (0.83) | (0.83) | (0.83) |
| IFI6 | MX1 | IFI44 | ISG15 | XAF1 | IFI27 | BST2 |
| | (0.87) | (0.86) | (0.81) | (0.77) | (0.74) | (0.65) |
| SIRPG | PTPN7 | CD3E | SLAMF6 | CD8A | TIGIT | MIR155HG |
| | (0.85) | (0.82) | (0.82) | (0.82) | (0.81) | (0.81) |
| KLF4 | ATF3 | FOS | EGR1 | NR4A1 | THBD | IER5 |
| | (0.68) | (0.68) | (0.67) | (0.53) | (0.53) | (0.5) |
| SLC4A1 | SLC12A3 | TMEM178A | VEGFA | HDAC6 | MME | ASB15 |
| | (0.66) | (0.65) | (0.65) | (0.64) | (0.63) | (0.63) |
| ADORA2A | IL10RB | PIK3CG | BATF | CSF2RB | SYK | IRF4 |
| | (0.69) | (0.67) | (0.65) | (0.65) | (0.65) | (0.65) |
| FABP1 | MME | ABCC2 | HDAC6 | RGN | HYAL1 | TPMT |
| | (0.87) | (0.86) | (0.81) | (0.8) | (0.79) | (0.79) |
| CCL3/L1 | TNFAIP3 | LILRB2 | LILRB1 | FCER1G | TLR8 | TNFRSF1B |
| | (0.82) | (0.81) | (0.8) | (0.8) | (0.8) | (0.79) |
| CFB | C3 | TIMP1 | SERPINA3 | FPR1 | FCGR2A | S100A9 |
| | (0.79) | (0.78) | (0.76) | (0.76) | (0.75) | (0.75) |
| MYBL1 | PIK3CG | IL2RG | CD45RA | BATF3 | SLAMF7 | CCR2 |
| | (0.64) | (0.64) | (0.63) | (0.62) | (0.62) | (0.62) |
| IL17RB | LRP2 | ABCC2 | CCL15 | AQP1 | SLC22A2 | TPMT |
| | (0.8) | (0.73) | (0.72) | (0.68) | (0.67) | (0.66) |

| Name of | Degree of | Degree of | Degree of | Degree of | Degree of | Degree of |
|---|---|---|---|---|---|---|
| Inform. | Correlation | Correlation | Correlation | Correlation | Correlation | Correlation |
| Gene | (in brackets) | (in brackets) | (in brackets) | (in brackets) | (in brackets) | (in brackets) |
| GNG11 | TEK | PPM1F | HEG1 | MMRN2 | CD34 | RHOJ |
| | (0.64) | (0.63) | (0.62) | (0.59) | (0.58) | (0.57) |
| IL7R | CD3D | TRAT1 | BTLA | CD3E | KLRB1 | IL2RG |
| | (0.83) | (0.83) | (0.83) | (0.83) | (0.83) | (0.82) |
| CXCL8 | S100A8 | PLAUR | CCL2 | TIMP1 | TREM1 | SOCS3 |
| | (0.63) | (0.63) | (0.62) | (0.62) | (0.62) | (0.62) |
| PLA1A | CX3CL1 | HLA-E | PRF1 | PSMB9 | B2M | TAP1 |
| | (0.65) | (0.64) | (0.6) | (0.59) | (0.59) | (0.59) |
| IGHG2 | IGHA1 | CD79A | POU2AF1 | IRF4 | FAM30A | IGHM |
| | (0.89) | (0.87) | (0.84) | (0.83) | (0.82) | (0.81) |
| KLRK1 | CD3E | LCK | CD96 | NLRC5 | CD247 | GZMK |
| | (0.87) | (0.87) | (0.87) | (0.87) | (0.87) | (0.87) |
| CXCR6 | CD8A | LCK | PTPN7 | SP140 | CCL5 | THEMIS |
| | (0.86) | (0.86) | (0.86) | (0.86) | (0.86) | (0.86) |
| CXCL13 | CD38 | BATF | AIM2 | CD72 | LAG3 | MIR155HG |
| | (0.76) | (0.76) | (0.76) | (0.76) | (0.76) | (0.76) |
| DEFB1 | CHCHD10 | ALDH3A2 | MME | TMEM178A | RXRA | SLC22A2 |
| | (0.65) | (0.65) | (0.62) | (0.61) | (0.61) | (0.6) |
| GNLY | GZMB | GBP5 | IL2RB | NLRC5 | CD45RB | HLA-E |
| | (0.72) | (0.72) | (0.72) | (0.71) | (0.71) | (0.7) |
| HLA-F | HLA-DPA1 | GBP5 | LCP2 | CCL5 | HLA-DRB3 | HLA-DMA |
| | (0.92) | (0.92) | (0.92) | (0.92) | (0.92) | (0.91) |
| BTG2 | BATF3 | NFKBIZ | TNFAIP3 | IL17RA | PNOC | SOCS3 |
| | (0.64) | (0.62) | (0.62) | (0.61) | (0.61) | (0.6) |
| CTLA4 | CD3E | BTLA | CD96 | LCP2 | ZAP70 | PSTPIP1 |
| | (0.88) | (0.87) | (0.87) | (0.87) | (0.87) | (0.86) |
| HLA-C | PSMB10 | NLRC5 | HLA-A | PSMB8 | GBP5 | HLA-DPA1 |
| | (0.76) | (0.76) | (0.76) | (0.75) | (0.75) | (0.74) |
| CASP3 | ARHGDIB | CD38 | CD84 | CGAS | CTSS | MS4A6A |
| | (0.76) | (0.76) | (0.76) | (0.76) | (0.75) | (0.75) |
| SH2D1B | IDO1 | CXCL9 | CARD16 | KLRD1 | CD247 | NLRC5 |
| | (0.62) | (0.62) | (0.62) | (0.61) | (0.61) | (0.6) |
| CXCL11 | PSMB9 | GBP5 | IRF1 | APOL1 | PSMB8 | CALHM6 |
| | (0.87) | (0.86) | (0.84) | (0.84) | (0.83) | (0.83) |
| GBP4 | TAP1 | GBP5 | HLA-E | IRF1 | PSMB8 | CALHM6 |
| | (0.86) | (0.86) | (0.85) | (0.84) | (0.83) | (0.83) |
| PHEX | CD8A | CD3D | CD3E | MIR155HG | SLAMF6 | CD7 |
| | (0.66) | (0.66) | (0.66) | (0.65) | (0.65) | (0.65) |
| ITGA4 | IL2RB | IL10RA | LCP2 | PSTPIP1 | CD3D | CIITA |
| | (0.71) | (0.71) | (0.71) | (0.71) | (0.7) | (0.7) |
| LCN2 | NNMT | C3 | MEGF11 | CXCL2 | S100A8 | ADAMTS1 |
| | (0.76) | (0.75) | (0.72) | (0.71) | (0.71) | (0.71) |
| HLA-DPB1 | HLA-DMB | IL2RB | LCP2 | PTPRC | IL10RA | INPP5D |
| | (0.93) | (0.93) | (0.92) | (0.92) | (0.92) | (0.92) |
| XCL1/2 | IDO1 | GBP5 | CIITA | NKG7 | HLA-E | HLA-DRA |
| | (0.79) | (0.79) | (0.78) | (0.78) | (0.78) | (0.78) |
| COL4A1 | OSMR | CD276 | MMP14 | LIF | MYC | ADAMTS1 |
| | (0.66) | (0.65) | (0.65) | (0.64) | (0.64) | (0.64) |
| PLAAT4 | STAT1 | PSMB9 | PSMB10 | PSME1 | WARS | TAP2 |
| | (0.85) | (0.84) | (0.83) | (0.83) | (0.82) | (0.81) |
| MCM6 | CD4 | ST8SIA4 | LILRB4 | LAIR1 | CD45R0 | CD84 |
| | (0.68) | (0.68) | (0.68) | (0.68) | (0.68) | (0.67) |
| CD69 | SP140 | LCK | THEMIS | TRAT1 | IL2RG | PTPRC |
| | (0.86) | (0.85) | (0.85) | (0.85) | (0.84) | (0.84) |
| CD40LG | IKZF1 | CD3E | IL2RG | CD5 | STAT4 | CD28 |
| | (0.8) | (0.8) | (0.8) | (0.8) | (0.8) | (0.8) |
| IL18 | MS4A4A | CD68 | CD163 | MS4A6A | IFNGR2 | LTF |
| | (0.63) | (0.63) | (0.63) | (0.62) | (0.61) | (0.61) |
| PECAM1 | VWF | CAV1 | ACVRL1 | CDH13 | RHOJ | RASIP1 |
| | (0.61) | (0.61) | (0.6) | (0.6) | (0.6) | (0.59) |
| CD2 | THEMIS | IKZF1 | SP140 | SLAMF6 | IL2RB | GZMA |
| | (0.92) | (0.92) | (0.91) | (0.91) | (0.91) | (0.91) |
| CD80 | LCP2 | IL10RA | HLA-DRB3 | CCR5 | PTPRC | LST1 |
| | (0.67) | (0.67) | (0.66) | (0.66) | (0.66) | (0.66) |
| SIGIRR | TPMT | SLC22A2 | LRP2 | ABCC2 | APOE | NOX4 |
| | (0.64) | (0.63) | (0.63) | (0.63) | (0.61) | (0.61) |
| CCL18 | CMKLR1 | CD68 | JAK3 | LY96 | ARHGDIB | IMPDH1 |
| | (0.71) | (0.71) | (0.7) | (0.7) | (0.69) | (0.69) |
| CD160 | TIGIT | FASLG | IL2RB | NKG7 | CXCR3 | SLAMF7 |
| | (0.69) | (0.69) | (0.69) | (0.69) | (0.69) | (0.69) |
| NOS3 | TM4SF1 | MCAM | | | | |
| | (0.62) | (0.6) | | | | |
| SERPINE1 | FCGR2A | MS4A4A | SLC11A1 | TLR2 | FCGR1A | TIMP1 |
| | (0.63) | (0.63) | (0.63) | (0.62) | (0.61) | (0.61) |
| FOXP3 | BTLA | IL16 | POU2AF1 | CD3E | PIK3CG | TRAT1 |
| | (0.63) | (0.63) | (0.63) | (0.63) | (0.62) | (0.62) |
| MYB | PSTPIP1 | CD3G | SELPLG | CD3D | IL2RA | CD3E |
| | (0.65) | (0.65) | (0.65) | (0.65) | (0.65) | (0.64) |
| HLA-B | HLA-A | PSMB8 | HLA-DRB3 | TAP2 | HLA-DMA | HLA-DPA1 |
| | (0.92) | (0.91) | (0.91) | (0.91) | (0.91) | (0.91) |
| CDH5 | CDH13 | PALMD | VEGFC | ACVRL1 | MCAM | RHOJ |
| | (0.64) | (0.62) | (0.62) | (0.61) | (0.61) | (0.6) |
| CD8B | GZMK | NLRC5 | CD3D | PTPN7 | CD7 | CD3E |
| | (0.74) | (0.74) | (0.74) | (0.74) | (0.74) | (0.74) |
| SOD2 | TIMP1 | HIF1A | CXCL1/2 | MEGF11 | LTF | IFNGR2 |
| | (0.71) | (0.7) | (0.69) | (0.69) | (0.69) | (0.67) |
| PRDM1 | CSF2RB | SLAMF7 | CCR2 | CD79A | IL17RA | AIM2 |
| | (0.82) | (0.82) | (0.82) | (0.81) | (0.81) | (0.81) |
| IFI6 | IFITM1 | MX2 | IRF7 | IFITM3 | SP100 | STAT1 |
| | (0.63) | (0.62) | (0.61) | (0.51) | (0.5) | (0.5) |
| SIRPG | PSTPIP1 | CD3G | SP140 | LCK | CD96 | CD3D |
| | (0.81) | (0.81) | (0.81) | (0.81) | (0.8) | (0.8) |
| ADORA2A | POU2AF1 | CD38 | SP140 | IL17RA | LCK | NLRC5 |
| | (0.64) | (0.64) | (0.64) | (0.64) | (0.63) | (0.63) |
| FABP1 | NOX4 | ALDH3A2 | LRP2 | SLC22A2 | LAMP1 | ASB15 |
| | (0.79) | (0.78) | (0.78) | (0.76) | (0.75) | (0.74) |
| CCL3/L1 | FCGR3A/B | ICAM1 | GBP2 | SLAMF8 | CTSS | LST1 |
| | (0.79) | (0.78) | (0.78) | (0.78) | (0.78) | (0.77) |
| CFB | CXCL16 | LAIR1 | LILRB4 | LTF | FCGR1A | ALOX5 |
| | (0.75) | (0.74) | (0.74) | (0.74) | (0.74) | (0.74) |
| MYBL1 | CASP1 | IL16 | IL2RB | PTPRC | CSF2RB | THEMIS |
| | (0.61) | (0.61) | (0.61) | (0.61) | (0.6) | (0.6) |
| IL17RB | GAPDH | MME | HNF1A | RGN | CXCL14 | LAMP1 |
| | (0.6) | (0.59) | (0.59) | (0.59) | (0.59) | (0.59) |

| Name of | Degree of | Degree of | Degree of | Degree of | Degree of | Degree of |
|---|---|---|---|---|---|---|
| Informative | Correlation | Correlation | Correlation | Correlation | Correlation | Correlation |
| Gene | (in brackets) | (in brackets) | (in brackets) | (in brackets) | (in brackets) | (in brackets) |
| IL7R | THEMIS | STAT4 | CD5 | PTPRC | LCK | CXCR4 |
| | (0.82) | (0.82) | (0.82) | (0.81) | (0.81) | (0.81) |
| CXCL8 | C5AR1 | IFITM2 | ICAM1 | NNMT | FPR1 | AHR |
| | (0.61) | (0.6) | (0.6) | (0.6) | (0.6) | (0.59) |
| IGHG2 | XBP1 | PNOC | FGFBP2 | CCR2 | BATF3 | CD27 |
| | (0.79) | (0.72) | (0.71) | (0.71) | (0.71) | (0.7) |
| KLRK1 | TIGIT | NKG7 | CD3D | CD3G | PRF1 | CXCR3 |
| | (0.86) | (0.86) | (0.86) | (0.86) | (0.85) | (0.85) |
| CXCR6 | CD3G | CD84 | LCP2 | PTPN22 | IKZF1 | CD45R0 |
| | (0.85) | (0.85) | (0.85) | (0.85) | (0.85) | (0.84) |
| CXCL13 | LILRB4 | ICOS | ST8SIA4 | IL21R | CXCR4 | JAK3 |
| | (0.76) | (0.76) | (0.76) | (0.75) | (0.75) | (0.75) |
| GNLY | PSMB9 | GBP1 | LST1 | HLA-DRB3 | CXCL10 | CIITA |
| | (0.7) | (0.7) | (0.7) | (0.7) | (0.7) | (0.7) |
| HLA-F | NKG7 | PSMB8 | CIITA | PTPN7 | CD8A | PSMB10 |
| | (0.91) | (0.9) | (0.9) | (0.9) | (0.9) | (0.9) |
| BTG2 | IL10RB | IL4R | IGHG4 | IGKC | IGLC1 | IGHG3 |
| | (0.6) | (0.6) | (0.6) | (0.59) | (0.59) | (0.59) |
| CTLA4 | TIGIT | CD45R0 | CD84 | AIM2 | INPP5D | ST8SIA4 |
| | (0.86) | (0.86) | (0.86) | (0.86) | (0.86) | (0.86) |
| HLA-C | GBP1 | CALHM6 | HLA-DMA | CXCL9 | NKG7 | PRF1 |
| | (0.74) | (0.73) | (0.73) | (0.73) | (0.73) | (0.73) |
| CASP3 | PTPN2 | CD47 | LAIR1 | IRF4 | ISG20 | CD86 |
| | (0.75) | (0.75) | (0.75) | (0.75) | (0.75) | (0.75) |
| SH2D1B | HLA-E | NKG7 | CTSW | CIITA | GZMB | IL2RB |
| | (0.6) | (0.6) | (0.6) | (0.59) | (0.59) | (0.59) |
| CXCL11 | TAP2 | HLA-E | APOL2 | STAT1 | GBP2 | PRF1 |
| | (0.83) | (0.83) | (0.83) | (0.82) | (0.81) | (0.81) |
| GBP4 | APOL1 | APOL2 | TAP2 | STAT1 | NLRC5 | GBP2 |
| | (0.83) | (0.83) | (0.83) | (0.82) | (0.82) | (0.8) |
| PHEX | IL2RB | SP140 | CXCR3 | IL21R | LCK | CD3G |
| | (0.65) | (0.64) | (0.64) | (0.64) | (0.64) | (0.64) |
| ITGA4 | IL2RG | LCK | IL16 | HLA-DPA1 | PTPN6 | CD96 |
| | (0.7) | (0.7) | (0.69) | (0.69) | (0.69) | (0.69) |
| LCN2 | CXCL16 | FCGR2A | LIF | NFKBIZ | HIF1A | FPR1 |
| | (0.69) | (0.69) | (0.68) | (0.68) | (0.67) | (0.66) |
| HLA-DPB1 | IL2RG | CCL5 | NLRC5 | CD48 | CD45R0 | PSMB9 |
| | (0.91) | (0.9) | (0.9) | (0.9) | (0.9) | (0.9) |
| XCL1/2 | CTSW | CXCR3 | GZMA | TRDC | SLAMF7 | PIK3CG |
| | (0.78) | (0.78) | (0.78) | (0.77) | (0.77) | (0.77) |
| COL4A1 | ITGB6 | HIF1A | FKBP1A | BAX | TNFRSF1A | SERPING1 |
| | (0.64) | (0.63) | (0.62) | (0.62) | (0.62) | (0.62) |
| PLAAT4 | HLA-A | GBP2 | CD74 | IRF1 | HLA-E | CALHM6 |
| | (0.81) | (0.81) | (0.81) | (0.8) | (0.8) | (0.79) |
| MCM6 | TLR8 | IFNAR2 | ALOX5 | CASP1 | IL2RA | PTPN7 |
| | (0.67) | (0.67) | (0.67) | (0.66) | (0.66) | (0.66) |
| CD69 | CD48 | PSTPIP1 | BTLA | LCP2 | TNFAIP3 | IL16 |
| | (0.84) | (0.84) | (0.84) | (0.84) | (0.83) | (0.83) |
| CD40LG | LCK | ZAP70 | CD48 | PIK3CG | CD3D | SP140 |
| | (0.8) | (0.8) | (0.79) | (0.79) | (0.79) | (0.79) |
| IL18 | TLR2 | S100A8 | CMKLR1 | IL2RA | TIMP1 | CASP4 |
| | (0.61) | (0.61) | (0.61) | (0.61) | (0.61) | (0.6) |
| CD2 | CCL5 | PSTPIP1 | CXCR3 | IL2RG | IL16 | CD5 |
| | (0.91) | (0.91) | (0.9) | (0.9) | (0.9) | (0.9) |
| CD80 | CD72 | SLAMF8 | IL2RB | TAP2 | PSTPIP1 | CD45RB |
| | (0.66) | (0.66) | (0.66) | (0.66) | (0.65) | (0.65) |
| SIGIRR | ASB15 | HYAL1 | SLC12A3 | | | |
| | (0.6) | (0.6) | (0.6) | | | |
| CCL18 | FPR1 | S100A9 | SLAMF8 | CD4 | ALOX5 | ADAMDEC1 |
| | (0.69) | (0.69) | (0.69) | (0.69) | (0.68) | (0.68) |
| CD160 | NCR1 | THEMIS | CTSW | TRAT1 | CD247 | AIM2 |
| | (0.69) | (0.68) | (0.68) | (0.68) | (0.68) | (0.68) |
| SERPINE1 | FCGR3A/B | C1QB | NNMT | C5AR1 | FPR1 | SERPING1 |
| | (0.61) | (0.61) | (0.6) | (0.6) | (0.59) | (0.59) |
| FOXP3 | TLR9 | LCK | CD3G | BTK | CSF2RB | AIM2 |
| | (0.62) | (0.62) | (0.62) | (0.62) | (0.61) | (0.61) |
| MYB | SLAMF6 | SP140 | CD45R0 | CD72 | ST8SIA4 | MIR155HG |
| | (0.64 | (0.64) | (0.64) | (0.64) | (0.64) | (0.64) |
| HLA-B | PSMB10 | NKG7 | IL2RB | CALHM6 | CCL5 | GBP2 |
| | (0.91) | (0.91) | (0.9) | (0.9) | (0.9) | (0.9) |
| CD8B | IL2RB | GZMA | CD247 | ZAP70 | MIR155HG | CD3G |
| | (0.74) | (0.73) | (0.73) | (0.73) | (0.73) | (0.72) |
| SOD2 | TNFRSF1A | CXCL16 | C3 | PTX3 | IL6 | NNMT |
| | (0.67) | (0.67) | (0.67) | (0.66) | (0.66) | (0.66) |
| PRDM1 | CD38 | BTK | CD3E | SLAMF6 | XBP1 | CD27 |
| | (0.81) | (0.8) | (0.8) | (0.8) | (0.8) | (0.8) |
| SIRPG | LAG3 | IL2RB | CD7 | SH2D1A | CXCR3 | ISG20 |
| | (0.8) | (0.8) | (0.8) | (0.79) | (0.79) | (0.79) |
| ADORA2A | NFATC2 | MICB | IL27RA | FAM30A | AIM2 | NOD2 |
| | (0.63) | (0.63) | (0.63) | (0.62) | (0.62) | (0.62) |
| FABP1 | APOE | MAF | CCL15 | HNF1A | CHCHD10 | AQP1 |
| | (0.74) | (0.74) | (0.74) | (0.73) | (0.73) | (0.72) |
| CCL3/L1 | IRF1 | FCGR1A | MYD88 | LCP2 | C3AR1 | IFI30 |
| | (0.77) | (0.77) | (0.76) | (0.76) | (0.76) | (0.76) |
| CFB | TLR2 | CASP4 | SERPING1 | MS4A6A | IFI30 | IFNGR2 |
| | (0.73) | (0.73) | (0.72) | (0.72) | (0.72) | (0.71) |
| MYBL1 | ST8SIA4 | CD48 | HLA-DPA1 | TNF | SELPLG | INPP5D |
| | (0.6) | (0.6) | (0.6) | (0.6) | (0.6) | (0.6) |

| Name of | Degree of | Degree of | Degree of | Degree of | Degree of | Degree of | Degree of |
|---|---|---|---|---|---|---|---|
| Inform. | Correl. (in | Correl. (in | Correl. (in | Correl. (in | Correl. (in | Correl. (in | Correl. (in |
| Gene | brackets) | brackets) | brackets) | brackets) | brackets) | brackets) | brackets) |
| IL7R | PSTPIP1 | SP140 | INPP5D | SELPLG | TCF7 | TNFSF8 | CD247 |
| | (0.81) | (0.81) | (0.81) | (0.8) | (0.79) | (0.79) | (0.79) |
| IGHG2 | SLAMF7 | THEMIS | FCGR2B | PIK3CG | SP140 | ISG20 | CSF2 |
| | (0.7) | (0.68) | (0.68) | (0.67) | (0.66) | (0.66) | (0.66) |
| KLRK1 | PTPN7 | NFATC2 | CTSW | SH2D1A | CD7 | PTPN22 | PSTPIP1 |
| | (0.85) | (0.85) | (0.84) | (0.84) | (0.84) | (0.84) | (0.84) |
| CXCR6 | SLA | ST8SIA4 | PSTPIP1 | CXCR3 | MIR155HG | IL2RB | GZMK |
| | (0.84) | (0.84) | (0.84) | (0.84) | (0.84) | (0.84) | (0.84) |
| CXCL13 | BTLA | ISG20 | CD3E | BTK | CD45R0 | CD27 | CD3D |
| | (0.75) | (0.75) | (0.75) | (0.74) | (0.74) | (0.74) | (0.74) |
| GNLY | HLA-DPA1 | LILRB2 | TAP2 | CD247 | CD74 | ITGAX | CTSW |
| | (0.7) | (0.69) | (0.69) | (0.69) | (0.69) | (0.68) | (0.68) |
| HLA-F | PTPRC | TAP2 | HLA-A | CALHM6 | IL10RA | GZMA | IKZF1 |
| | (0.9) | (0.9) | (0.9) | (0.9) | (0.9) | (0.9) | (0.89) |
| CTLA4 | IL2RB | ICOS | PTPRC | SLA | JAK3 | SELPLG | IL16 |
| | (0.86) | (0.86) | (0.86) | (0.86) | (0.86) | (0.86) | (0.85) |
| HLA-C | IDO1 | GBP2 | IL2RB | CCL5 | STAT1 | LCP2 | C1QA |
| | (0.73) | (0.73) | (0.73) | (0.72) | (0.72) | (0.72) | (0.72) |
| CASP3 | DNMT1 | CD4 | CD45R0 | SLA | CASP1 | LILRB4 | SP140 |
| | (0.74) | (0.74) | (0.74) | (0.74) | (0.74) | (0.74) | (0.74) |
| SH2D1B | HLA-DPA1 | PIK3CG | MEOX1 | KIR3DL1 | GBP5 | SLAMF7 | HLA-DRB3 |
| | (0.58) | (0.58) | (0.58) | (0.58) | (0.57) | (0.57) | (0.57) |
| CXCL11 | PSME2 | NLRC5 | PSMB10 | HLA-A | NKG7 | CD74 | B2M |
| | (0.81) | (0.8) | (0.8) | (0.79) | (0.79) | (0.79) | (0.78) |
| GBP4 | HLA-DPA1 | NKG7 | PRF1 | PSME2 | CIITA | CD74 | PSMB10 |
| | (0.8) | (0.79) | (0.79) | (0.79) | (0.79) | (0.79) | (0.79) |
| PHEX | SH2D1A | PTPN22 | ZAP70 | CD27 | CD96 | PSTPIP1 | CD5 |
| | (0.64) | (0.63) | (0.63) | (0.63) | (0.63) | (0.63) | (0.63) |
| ITGA4 | SELPLG | IRF8 | NLRC5 | HLA-DRB3 | CD45RB | CD3E | CD45R0 |
| | (0.69) | (0.69) | (0.69) | (0.69) | (0.69) | (0.69) | (0.68) |
| LCN2 | ALOX5 | ITGB6 | VCAN | CSF3R | IFITM2 | OSMR | MYC |
| | (0.66) | (0.66) | (0.65) | (0.65) | (0.65) | (0.65) | (0.64) |
| HLA-DPB1 | IKZF1 | GBP5 | PTPN22 | GZMA | ZAP70 | CD4 | ST8SIA4 |
| | (0.9) | (0.89) | (0.89) | (0.89) | (0.89) | (0.89) | (0.89) |
| XCL1/2 | CXCL9 | CD247 | CD96 | CD74 | LCK | TBX21 | PTPRC |
| | (0.77) | (0.77) | (0.77) | (0.77) | (0.77) | (0.77) | (0.77) |
| COL4A1 | FN1 | MEGF11 | FCGR2A | STAT3 | CXCL1/2 | LTF | NFKBIZ |
| | (0.61) | (0.61) | (0.61) | (0.61) | (0.6) | (0.6) | (0.59) |
| PLAAT4 | GBP5 | IDO1 | HLA-DMA | B2M | CXCL10 | APOL1 | IL18BP |
| | (0.79) | (0.79) | (0.78) | (0.78) | (0.78) | (0.78) | (0.77) |
| MCM6 | PTPN6 | CTSS | MS4A6A | FCGR1A | ITGB2 | PIK3CD | LCP2 |
| | (0.66) | (0.66) | (0.66) | (0.66) | (0.66) | (0.66) | (0.66) |
| CD69 | SELPLG | INPP5D | IL2RB | PTPN7 | CD247 | CD5 | NFATC2 |
| | (0.83) | (0.83) | (0.82) | (0.82) | (0.82) | (0.82) | (0.82) |
| CD40LG | BTLA | CXCR3 | PTPRC | PSTPIP1 | CCR2 | INPP5D | SELPLG |
| | (0.77) | (0.77) | (0.77) | (0.77) | (0.77) | (0.76) | (0.76) |
| IL18 | ALOX5 | CXCL16 | PTPN2 | RNF149 | LAIR1 | SERPINA3 | CSF3R |
| | (0.6) | (0.6) | (0.6) | (0.6) | (0.59) | (0.59) | (0.59) |
| PECAM1 | BATF3 | NCR1 | PIK3CG | TGFBR2 | FCGR3A/B | CASP1 | APOL1 |
| | (0.55) | (0.55) | (0.55) | (0.55) | (0.54) | (0.54) | (0.54) |
| CD2 | CD3G | LCP2 | CD45R0 | PTPN22 | PTPRC | GZMK | CD8A |
| | (0.9) | (0.9) | (0.9) | (0.9) | (0.9) | (0.9) | (0.89) |
| CD80 | SLAMF7 | JAK3 | PSMB9 | HLA-DMA | PIK3CG | MIR155HG | LILRB1 |
| | (0.65) | (0.65) | (0.65) | (0.65) | (0.65) | (0.65) | (0.65) |
| CCL18 | TLR8 | CSF3R | MMP9 | IFI30 | C3AR1 | BTK | CTSS |
| | (0.68) | (0.68) | (0.67) | (0.67) | (0.67) | (0.67) | (0.66) |
| CD160 | CD96 | GZMA | SH2D1A | MIR155HG | CCL5 | HLA-E | LCK |
| | (0.68) | (0.68) | (0.67) | (0.67) | (0.67) | (0.67) | (0.67) |
| FOXP3 | CCR4 | CD96 | TNFSF14 | IKZF1 | CCR2 | IL27RA | TNFSF8 |
| | (0.61) | (0.61) | (0.61) | (0.61) | (0.61) | (0.61) | (0.61) |
| MYB | EZH2 | CD96 | CD28 | TIGIT | TNFSF8 | IL21R | CD5 |
| | (0.64) | (0.64) | (0.64) | (0.64) | (0.63) | (0.63) | (0.63) |
| HLA-B | LCP2 | PRF1 | HLA-DMB | IL10RA | CD8A | GBP1 | GZMA |
| | (0.9) | (0.89) | (0.88) | (0.88) | (0.88) | (0.88) | (0.88) |
| CD8B | CD96 | CXCR3 | NKG7 | FASLG | SH2D1A | FYN | HLA-E |
| | (0.72) | (0.72) | (0.72) | (0.71) | (0.71) | (0.71) | (0.71) |
| SOD2 | IFI30 | FCGR2A | BCL3 | NFKBIZ | CASP4 | SLPI | LIF |
| | (0.66) | (0.65) | (0.65) | (0.65) | (0.64) | (0.64) | (0.64) |
| PRDM1 | PIK3CG | ST8SIA4 | BATF3 | THEMIS | PTPN7 | IKZF1 | CD96 |
| | (0.8) | (0.8) | (0.8) | (0.79) | (0.79) | (0.79) | (0.78) |
| SIRPG | IL21R | AIM2 | ZAP70 | CD72 | CD38 | IKZF1 | ICOS |
| | (0.79) | (0.79) | (0.79) | (0.79) | (0.79) | (0.79) | (0.78) |
| ADORA2A | PTPN7 | TNFRSF4 | IL21R | PTGER4 | CARD16 | TGFB1 | SLAMF7 |
| | (0.62) | (0.62) | (0.62) | (0.62) | (0.62) | (0.62) | (0.62) |
| FABP1 | SLC12A3 | VEGFA | CXCL14 | RXRA | TMEM178A | SDC1 | AKR1C3 |
| | (0.71) | (0.71) | (0.67) | (0.66) | (0.63) | (0.62) | (0.62) |
| CCL3/L1 | LAIR1 | LILRB4 | GBP5 | IL10RA | NKG7 | HLA-DMA | TLR2 |
| | (0.76) | (0.76) | (0.76) | (0.76) | (0.76) | (0.75) | (0.75) |
| CFB | LY96 | C1QB | CD68 | FCER1G | CTSS | S100A8 | IFNAR2 |
| | (0.71) | (0.71) | (0.71) | (0.71) | (0.71) | (0.71) | (0.71) |
| MYBL1 | AOAH | NOD2 | CD3E | IL10RA | HLA-DRA | IRF4 | IKZF1 |
| | (0.6) | (0.6) | (0.6) | (0.6) | (0.6) | (0.6) | (0.59) |

In some embodiments, two or more genes are determined to be correlated if they exhibit similar expression patterns across a set of samples from transplant recipients, some of whom have experienced transplant rejection and some of whom have not experienced transplant rejection. In some embodiments, two or more genes are determined to be correlated when their expression levels increased or decreased to a similar extent in the same samples. Exemplary methods for clustering based on gene expression patterns are described, for example, in Oyelade, J. et al., Bioinform Biol Insights. 2016; 10: 237-253 which is hereby incorporated by reference in its entirety. In some embodiments, clustering is based on genes, samples, and/or other variables, and is performed using various clustering methods such as hierarchical clustering (HC), self-organizing maps (SOM), and/or K-means clustering.

In some embodiments, the plurality of genes associated with immune cell activation, organ-specific defense against pathogens, regulation of tissue and cellular processes, and/or transcription regulation may comprise 2-10, 11-20, 21-30, 31-40, 41-50, 51-60, 61-70, 71-80, 81-90, 91-100, 101-120, 121-150, 151-200, 201-250, 251-300, 301-400, 401-500, 501-600, 601-700, 701-800, 801-1000, or more, genes. In some embodiments, at least one gene of the plurality of genes may comprise a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1.

In some embodiments, at least one gene of the plurality of genes may comprise a gene that is determined to be correlated with a gene that is associated with immune cell activation, organ-specific defense against pathogens, regulation of tissue and cellular processes, or transcription regulation.

In some embodiments, the biomarker unit 310 may provide gene expression levels by testing a gene panel comprising one or more informative genes from a plurality of genes associated with immune cell activation, organ-specific defense against pathogens, regulation of tissue and cellular processes, and/or transcription regulation, utilizing a biological sample, such as FFPE renal allograft biopsy tissue, comprising nucleic acids. In some embodiments, the nucleic acids from the biological sample comprise mRNA. In some embodiments, the nucleic acids from the biological sample comprise total RNA. In some embodiments, the nucleic acids, e.g., total RNA, may be extracted from the biological sample, e.g., from tissue curls of an organ tissue sample. Various methods of extracting nucleic acids, such as mRNA or total RNA, are known in the art, e.g., methods as described in Sambrook et al. Molecular Cloning: A Laboratory Manual 4th edition (2014) Cold Spring Harbor Laboratory Press, Cold Spring Harbor, N.Y.; Ausubel, et al., Current Protocols in Molecular Biology (2010). Nucleic acid extraction may also be performed using commercial purification kits, buffer sets, and proteases in accordance to the manufacturers' instructions or any suitable method. Once the nucleic acids are extracted, they can be frozen or otherwise stored in a condition that maintains the integrity and prevents degradation and/or contamination of the nucleic acids, or used directly for downstream applications and analysis, such as analysis of gene expression levels of one or more informative genes. In some embodiments, gene expression levels may be determined by analyzing total RNA from the sample, e.g., using RNA-sequencing. In some embodiments, gene expression levels may be determined by analyzing mRNA from the sample. In some embodiments, the RNA may be fragmented and used as a template to synthesize cDNA. The cDNA may be then subjected to 3′-adenylation and 5′-end repair. Sequencing adaptors may be ligated onto the 3′-adenylation and 5-end repaired cDNA, and the adaptor-ligated cDNA may then be amplified prior to sequencing. In some embodiments, gene expression levels are determined by quantifying RNA levels, e.g., mRNA transcript levels, without amplification and/or reverse transcription to cDNA, e.g., using a gene expression platform such as the NanoString Technologies nCounter® system. In some embodiments, a gene expression platform may quantify mRNA transcript levels for one or more informative genes from the gene panel. As discussed in more detail below, the gene panel may be a subset of genes identified from a plurality of genes of the biological sample, associated with immune cell activation, organ-specific defense against pathogens, regulation of tissue and cellular processes, and/or transcription regulation. In some embodiments, a gene expression platform may be utilized that does not require instant preservation in RNA stabilization and storage reagents after sample collection, e.g., by using the same biopsy core from the routine histopathologic assessment. In some embodiments, a FFPE organ tissue sample, e.g., a renal allograft biopsy tissue sample, is obtained from routine clinical pathology practice and used for determining gene expression levels. In some embodiments, the FFPE organ tissue sample used for the determination of gene expression levels may be archived clinical samples, including older samples (e.g., 5, 6, 10, 13 years old, etc.). In some embodiments, a gene expression platform, such as the NanoString Technologies nCounter® system, may be used to develop gene expression signatures for transplant rejection diagnosis in recipients of transplants.

In some embodiments, the database 320 may store various characteristics, such as gene expression levels of a plurality of genes in a biological sample, e.g., an organ tissue sample, from a transplant recipient that may be informative with regards to determining the status of the transplant, or transplant lesion scores, e.g., organ transplant lesion scores, that may have been assigned by one or more pathologists, e.g., renal pathologists, upon histopathological evaluation of a biological sample, e.g., an organ tissue sample, from a kidney transplant recipient. In some embodiments, one or more lesion scores may be stored in the database 320. In some embodiments, one or more rejection classifications may be stored in the database 320 that may have been assigned by one or more pathologists, e.g., renal pathologists, based on the one or more lesion scores that were assigned upon histopathological evaluation of a biological sample, e.g., an organ tissue sample. In some embodiments, one or more rejection classifications may be stored in the database 320 that may have been assigned by one or more pathologists, e.g., renal pathologists, based on the one or more lesion scores alone or in combination with additional lab test results. In some embodiments, one or more rejection classifications may be stored in the database 320 that may have been assigned based on the one or more lesion scores alone or in combination with additional lab test results, in accordance with guidelines for the classification of human transplants, e.g., Banff 2019 classification guidelines for human organ transplants (Mengel et al. (2019) Am J Transplant. 2020 20: 2305-2317.)

In some embodiments, the data stored in the database 320 may comprise a discovery dataset from biological samples of a discovery cohort of transplant recipients, e.g., organ transplant recipients, and a validation dataset from biological samples of a validation cohort of transplant recipients, e.g., organ transplant recipients. In some embodiments, one or more of the transplant recipients (of the discovery dataset, validation dataset, or both) may have received an organ transplant comprising one or more of: a kidney transplant, a heart transplant, a lung transplant, a pancreas transplant, a liver transplant, an intestinal transplant, or a vascularized composite allograft transplant. In some embodiments, one or more of the transplant recipients may have received a transplant that is an allograft or a xenograft. In some embodiments, the discovery dataset may comprise gene expression levels of a plurality of genes and rejection classifications for the discovery cohort biological samples. In some embodiments, the discovery dataset may comprise gene expression levels of a plurality of genes associated with immune cell activation, organ-specific defense against pathogens, regulation of tissue and cellular processes, and/or transcription regulation. In some embodiments, the validation dataset may comprise gene expression levels of a plurality of genes and rejection classifications for the validation cohort biological samples. The discovery dataset may also comprise rejection classifications for the discovery cohort biological samples. In some embodiments, the validation dataset may comprise gene expression levels of a plurality of genes associated with immune cell activation, organ-specific defense against pathogens, regulation of tissue and cellular processes, and/or transcription regulation.

In some embodiments, the discovery dataset may comprise data from biological samples, obtained from transplant recipients, exhibiting diverse histologic findings (e.g., different types of rejection, non-diagnostic for rejection from both renal allograft and native kidney, etc.). For example, in some embodiments, at least some of the rejection classifications of the discovery dataset may comprise ABMR. In some embodiments, at least some of the rejection classifications of the discovery dataset may comprise TCMR. In some embodiments, at least some of the rejection classifications of the discovery dataset may comprise mixed ABMR+TCMR. In some embodiments, at least some of the rejection classifications of the discovery dataset may comprise no rejection.

Embodiments of the disclosure may comprise systems and methods capable of differentiating conditions of inflammation associated with renal allograft rejection from conditions caused by other pathologic conditions unrelated to rejection (including various types of viral or bacterial infection, or various types of glomerulopathy). In some embodiments, the discovery dataset may include data from biological samples, e.g., organ tissue samples such as biopsy samples, that originate from both a native organ (e.g., native kidney(s)), an organ transplant (e.g., kidney transplant), and exhibit various types of inflammation, such as cytomegalovirus (CMV) or BK virus (BKV) nephropathy, acute pyelonephritis, diabetic nephropathy, etc.

The machine-learning model 330 may be trained to generate a plurality of sets of weights to be used by system 100 in generating one or more probability rejection scores and assigning a predictive rejection classification. In some embodiments, the machine-learning model 330 may be trained to analyze gene expression levels of a discovery dataset for associations with rejection classifications in the discovery dataset. In some embodiments, the machine-learning model 330 may narrow down the set of genes (to which sets of weights are generated) by identifying a subset of genes (from the plurality of informative genes of the discovery dataset) based on the fitting process disclosed herein. In some embodiments, the machine-learning model 330 may generate a plurality of sets of weights for the subset of genes.

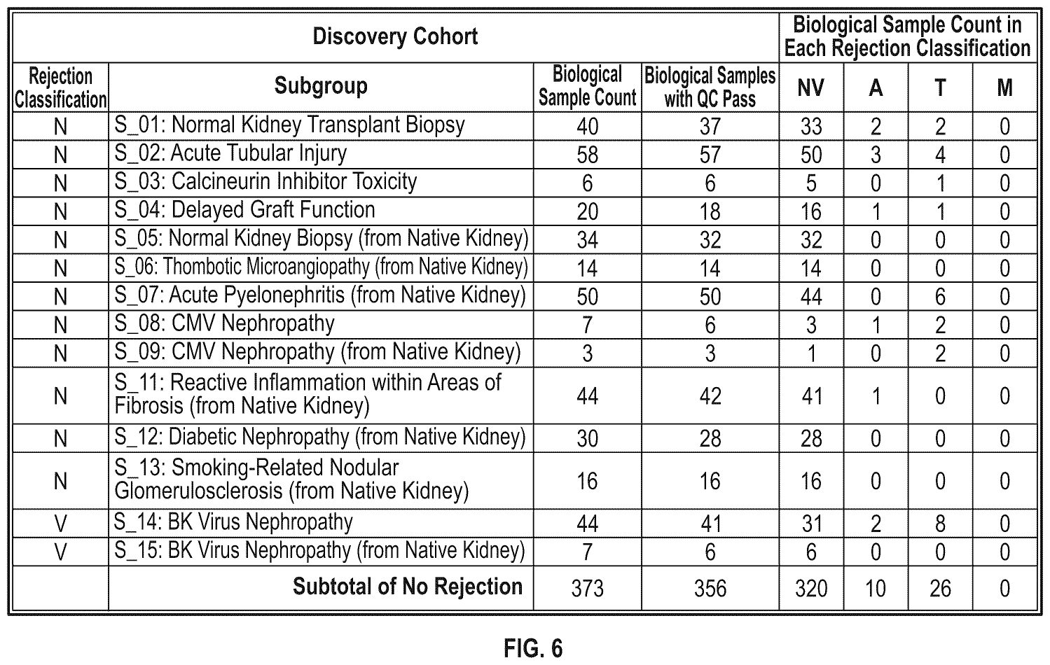

FIG. 4 illustrates a flow chart of an example method performed by a machine-learning model, according to embodiments of the disclosure. In some embodiments, the plurality of sets of weights for the plurality of informative genes may be from a machine-learning model trained to perform one or more steps of method 400. In some embodiments, method 400 may comprise receiving a discovery dataset from, e.g., a database 320, in step 402. In some embodiments, the discovery dataset may comprise gene expression levels and associated rejection classifications for biological samples of a discovery cohort of transplant recipients, e.g., organ transplant recipients. In some embodiments, the discovery dataset may be data obtained by one or more units, such as biomarker unit 310.

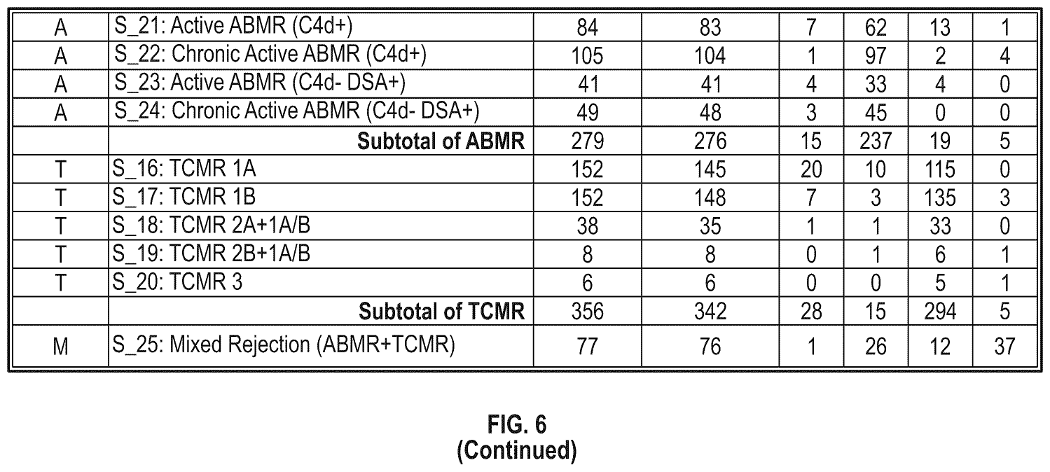

For example, as shown in FIG. 5, the discovery dataset may comprise data related to, e.g., histopathological evaluation, lesion scoring, and so forth, for a plurality of transplant tissue samples, e.g., organ tissue biopsies, of a discovery cohort. In some embodiments, the system may perform quality control, for example, using predetermined thresholds not to be exceeded or expected ranges, such that data from biological samples that do not meet certain criteria, for example, based on a predetermined threshold, may not be included in any subsequent evaluation or calculation. In some embodiments, one example criterion may include identifying genes that have an expected range or do not exceed a predetermined threshold, for example, related to gene normalization quality control, e.g., when identifying housekeeping genes for gene normalization. In certain embodiments, another example criterion may include identifying genes that fulfill certain performance criteria, for example, related to assay efficiency, limiting detection or minimum detection threshold, for example, setting a target threshold for detecting and quantifying targets, e.g., a performance criterion of detecting a certain percentage of probes that target informative genes, such as a detection threshold of 62%.

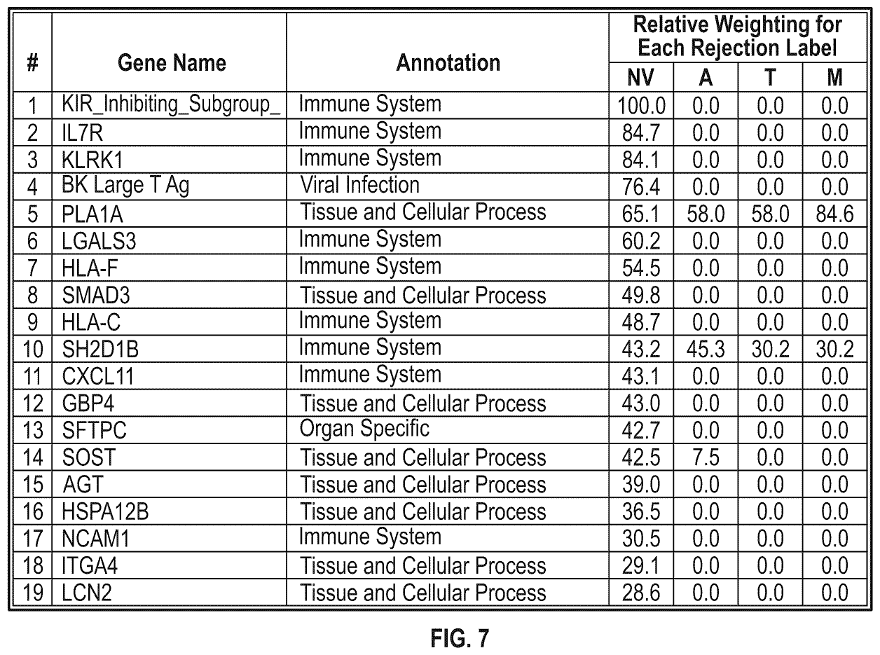

FIG. 6 illustrates a table of example discovery dataset, according to embodiments of the disclosure. In some embodiments, the biological samples of the discovery cohort may include biological samples assigned with a rejection classification of (either active or chronic) ABMR. In some embodiments the biological samples of the discovery cohort may include biological samples assigned with a rejection classification of (various grades of acute) TCMR. In some embodiments, the biological samples of the discovery cohort may include biological samples assigned with a rejection classification of mixed ABMR+TCMR. In some embodiments, the biological samples of the discovery cohort may include biological samples with a rejection classification of “no rejection” (having various histologic findings nondiagnostic for any type of rejection or lacking one or more histologic findings diagnostic for any type of rejection). In some embodiments, the “no rejection” biological samples of the discovery dataset may comprise biological samples with or without various types of inflammation from a native organ, e.g., native kidney. In some embodiments, the “no rejection” biological samples of the discovery dataset may comprise biological samples, e.g., renal allograft biopsies, without inflammation or with inflammation, e.g., viral infection associated inflammation (CMV or BKV).

In some embodiments, at least one of the plurality of informative genes of the discovery dataset may be associated with one or more of: immune cell activation, organ-specific defense against pathogens, regulation of tissue and cellular processes, or transcription regulation.

In some embodiments, at least one gene of the plurality of informative genes of the discovery dataset may comprise a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1.

In some embodiments, at least one gene of the plurality of informative genes of the discovery dataset may comprise a gene that exhibits a correlation of at least 0.6 or 60% with a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1.

Returning to FIG. 4, in step 404, gene expression levels of the discovery dataset may be analyzed for associations with the rejection classifications in the discovery dataset. For example, in some embodiments, a multinomial regression model may be used to fit the gene expression levels of the discovery dataset to determine whether there is an association with the corresponding rejection classification. In some embodiments, the multinomial regression model may estimate coefficients using a regularized likelihood. In some embodiments, the gene expression levels may be analyzed by detecting and/or quantifying nucleic acids or RNA from the biological samples of the discovery cohort.

In some embodiments, the expression levels of one or more genes from the plurality of genes of the discovery dataset may be normalized relative to gene expression levels of one or more reference genes. In some embodiments, normalization may be performed using housekeeping genes. In some embodiments, normalization may be performed using normalization quality control metrics.

In step 406, a subset of genes from the plurality of genes of the discovery dataset may be identified. In one example embodiment of the disclosure, the plurality of genes of the discovery dataset represented more than 700 genes, and a subset of less than 200 genes was identified for subsequent predictive rejection classification.

In step 408, the machine-learning model 330 may generate a plurality of sets of weights for the subset of genes (from step 406). The plurality of sets of weights may be generated based on the associations between the gene expression levels of the discovery dataset and the rejection classifications of the discovery dataset. In some embodiments, each set of weights may be associated with one gene of the subset of genes. The plurality of sets of weights may be calculated using a prediction model such as lasso regularized regression, elastic net random forests, gradient boosted machine, k nearest neighbors, or support vector machine. The process performed by the prediction model may involve fitting by cross-validation (e.g., a 10-fold cross-validation) to determine one or more hyper-parameter.

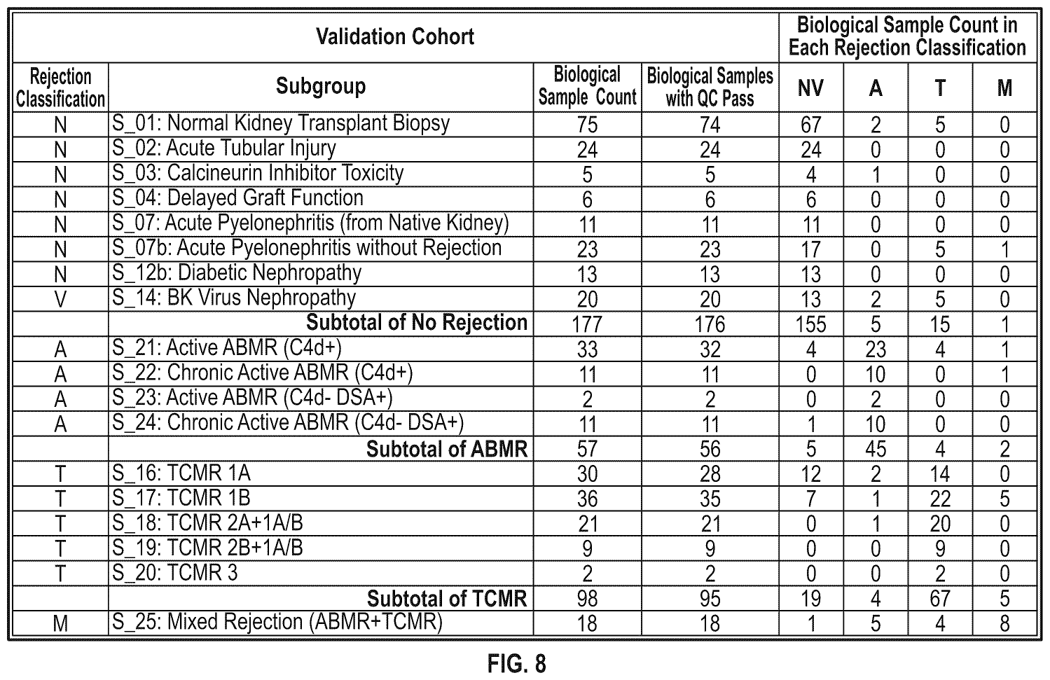

FIG. 7 illustrates a table of example sets of weights for a subset of genes, according to embodiments of the disclosure. For example, the KIR_Inhibiting_Subgroup_1 gene may have weights of 100, 0, 0, and 0 relative to other genes of the plurality of informative genes for the different rejection labels: no rejection, ABMR, TCMR, and mixed ABMR+TCMR, respectively. As another example, the PLA1A gene may have weights of 65.1, 58.0, 58.0 and 84.6 relative to other genes of the plurality of informative genes for the different rejection labels: no rejection, ABMR, TCMR, and mixed ABMR+TCMR, respectively. As shown in the table, in some embodiments, each set of weights comprises a weight for a corresponding rejection label.

Embodiments of the disclosure may include training the machine-learning model. The machine-learning model may be trained by using a discovery dataset from biological samples of a discovery cohort of transplant recipients, e.g., organ transplant recipients. The machine-learning model may be trained to receive the discovery dataset, analyze gene expression levels of the discovery dataset, identify a subset of genes, and generate a plurality of sets of weights for the subset of genes.

The machine-learning model may be validated using a validation cohort to determine whether it was trained according to certain criteria, e.g., diagnosis accuracy. In some embodiments, a diagnosis accuracy being greater than a predetermined value may be one criterion. In some embodiments, the diagnosis accuracy may be determined based on a comparison of one or more rejection classifications in a dataset and one or more computer-determined predictive rejection classifications.

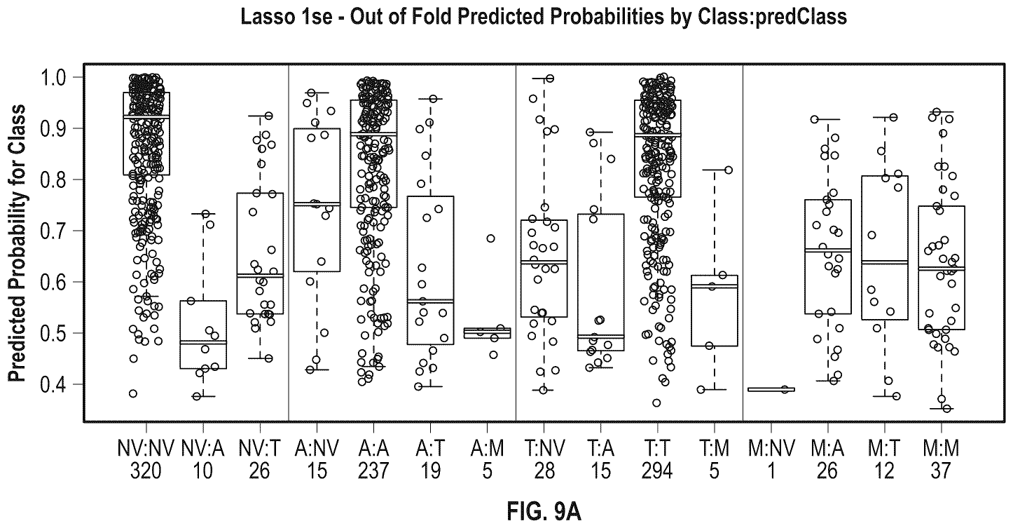

In some embodiments, a dataset used for validating the machine-learning model may be a validation dataset or cohort, as shown in FIGS. 5 and 8. In some embodiments, the validation dataset may comprise data for hundreds or thousands of biological samples, e.g., organ tissue samples such as biopsy samples, of a validation cohort. In some embodiments, the validation dataset may be evaluated on the basis of various quality control metrics to assess and ensure consistency, reliability and reproducibility in predicting rejection classifications that must be fulfilled for subsequent use in validating the machine-learning model. In some embodiments, the biological samples of the validation cohort may include biological samples assigned with a rejection classification of ABMR. In some embodiments, the biological samples of the validation cohort may include biological samples assigned with a rejection classification of TCMR. In some embodiments, the biological samples of the validation cohort may include biological samples assigned with a rejection classification of mixed ABMR+TCMR. In some embodiments, the biological samples of the validation cohort may include biological samples assigned with a rejection classification of “no rejection.”

The computer-determined predictive rejection classifications may be determined for a validation dataset using probability rejection scores generated based on the expression levels of a plurality of genes with a plurality of sets of weights generated by the machine-learning model from biological samples of the validation cohort. In some embodiments, the computer-determined predictive classifications will be acceptable if diagnosis accuracy exceeds a predetermined value. In some embodiments, the predetermined value may be 60%, 70%, 80%, or 90%. The diagnosis accuracy may represent the percentage of the predictive rejection classifications in the validation dataset that match the computer-determined predictive rejection classifications (determined from the validation dataset).

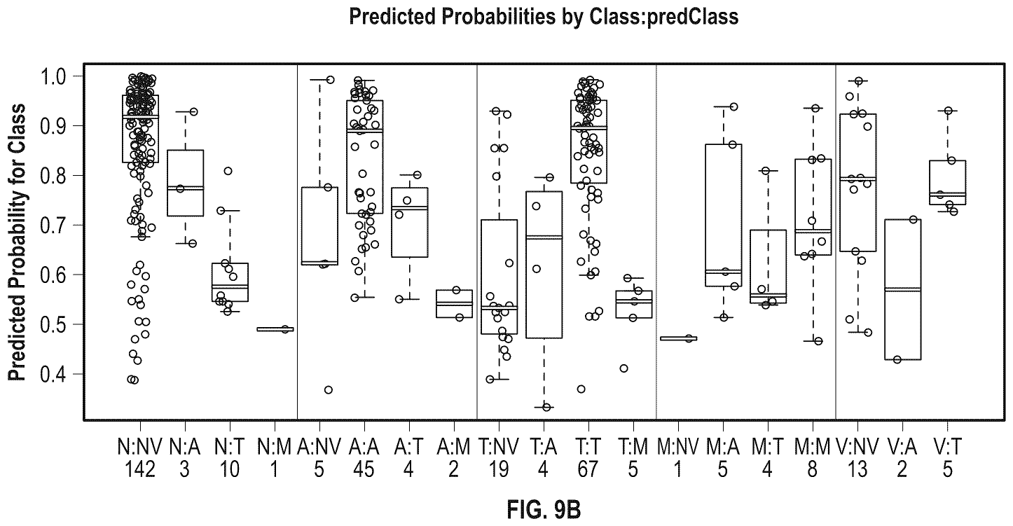

In some embodiments, the diagnosis accuracy may be different for different predictive rejection classifications and/or different datasets. FIG. 9A shows the diagnosis accuracy for the discovery dataset, according to embodiments of the disclosure. For example, for the discovery dataset, the overall diagnosis accuracy may be 84.6%. In some embodiments, the performance characteristics, for example, the sensitivity and the specificity, of the disclosed systems and methods may be different for different predictive rejection classifications and/or different datasets. In some example embodiments, the sensitivity and specificity of the disclosed systems and methods were 93.7% and 89.9%, respectively, regardless of the predictive rejection classification. In some example embodiments, the sensitivity for ABMR or TCMR predictive rejection classifications was above 85%. In some example embodiments, the sensitivity for a predictive rejection classification of mixed ABMR+TCMR was approximately 50%. In some example embodiments, the specificity for each of the three different types of rejections (e.g., ABMR, TCMR, mixed ABMR+TCMR) was above 90%.

In some embodiments, the performance characteristics, for example, the diagnosis accuracy, sensitivity, specificity, of the disclosed systems and methods may be different for different predictive rejection classifications and/or different datasets. FIG. 9B shows the diagnosis accuracy for the validation dataset, according to example embodiments of the disclosure. In some example embodiments, the diagnosis accuracy was 79.7%, while sensitivity and specificity were 85.2% and 88.1%, respectively, regardless of the predictive rejection classification. In some example embodiments, sensitivity was 80.4%, 70.5%, and 44.4% for ABMR, TCMR, and mixed ABMR+TCMR predictive rejection classifications, respectively. In some embodiments, the specificity for each of the three different types of rejections (e.g., ABMR, TCMR, mixed ABMR+TCMR) may be above 90%. The high specificity for predicting transplant rejection shows the potential of the disclosed systems and methods to successfully and reproducibly differentiate transplant rejection from other, rejection-unrelated conditions that might present with clinical parameters that are similar to rejection-associated parameters, such conditions including acute and/or chronic inflammatory diseases and/or systemic infections. In a demonstration of improved diagnostic accuracy, the disclosed systems and methods may be useful in differentiating transplant rejection from inflammatory and/or infectious conditions unrelated to rejection, e.g., diabetic nephropathy, acute pyelonephritis, BK virus nephropathy) in transplant recipients who present with some clinical concern and/or clinical parameters suggestive of transplant rejection but who actually suffered from inflammatory and/or infectious conditions unrelated to rejection by accurately assigning a predictive rejection classification of “no rejection.”

If the machine-learning model is not trained adequately (e.g., the diagnosis accuracy is not greater than the predetermined value), the training data (e.g., discovery dataset) may be revised to provide feedback to the model. In some embodiments, the output of the machine-learning model between training iterations may be evaluated by a medical expert or treating physician to determine which data in the training data should be revised. The treating physician or medical expert can revise certain data in areas of potential improvement, such as the weights of the expression levels of the plurality of genes.

### Example Administration of Immunosuppressive Therapy

Immunosuppressive therapy generally refers to the administration of an immunosuppressant or other therapeutic agent that suppresses immune responses to a transplant recipient. Example immunosuppressant agents may include, for example, calcineurin inhibitors, mTor inhibitors, anticoagulants, antimalarials, cardiovascular agents including but not limited to ACE inhibitors and β-blockers, non-steroidal anti-inflammatory drugs (NSAIDs), aspirin, azathioprine, B7RP-1-fc, brequinar sodium, campath-1H, celecoxib, chloroquine, corticosteroids, coumadin, cyclophosphamide, cyclosporin A, DHEA, deoxyspergualin, dexamethasone, diclofenac, dolobid, etodolac, everolimus, FK778, feldene, fenoprofen, flurbiprofen, heparin, hydralazine, hydroxychloroquine, CTLA-4 or LFA3 immunoglobulin, ibuprofen, indomethacin, ISAtx-247, ketoprofen, ketorolac, leflunomide, meclophenamate, mefenamic acid, mepacrine, 6-mercaptopurine, meloxicam, methotrexate, mizoribine, mycophenolate mofetil, naproxen, oxaprozin, Plaquenil, NOX-100, prednisone, methylprednisolone, rapamycin (sirolimus), sulindac, tacrolimus (FK506), thymoglobulin, tolmetin, tresperimus, UO126, and antibodies including, for example, alpha lymphocyte antibodies, adalimumab, anti-CD3, anti-CD25, anti-CD52, anti-IL2R, anti-TAC antibodies, basiliximab, daclizumab, etanercept, hu5C8, infliximab, OKT4, natalizumab, and any combination thereof. Immunosuppressive therapy may be adjusted in response to the classification of the status of a transplant as experiencing “no rejection,” ABMR, TCMR, or mixed ABMR+TCMR rejection. For example, in response to the classification of the status of a transplant as experiencing TCMR, bolus steroid treatment may be initiated, or maintenance immunosuppressive therapy may be increased with respect to dosage and/or frequency. In response to the classification of the status of a transplant as experiencing ABMR, for example, plasmapheresis or intravenous immunoglobulin (IVIg) may be initiated.

In some embodiments, no change in the status of a transplant (e.g., as indicated by no change in the predictive rejection classification) may indicate no need to adjust immunosuppressive therapy being administered to the transplant recipient, or that the immunosuppressive therapy being administered may be maintained. The decision to maintain immunosuppressive therapy being administered to a transplant recipient may be based on additional clinical factors, such as, for example, the health, age, comorbidities of the transplant recipient.

In some embodiments, adjustment of immunosuppressive therapy includes changing the type, form, or frequency of immunosuppressive therapy or other transplant-related therapy being administered to the transplant recipient. In some embodiments, where the transplant recipient is not receiving immunosuppressive therapy, the methods of the present disclosure may indicate a need to begin administering immunosuppressive therapy to the transplant recipient.

Other transplant-related therapies include treatments or therapies besides transplantation or immunosuppressive therapy that are administered to a transplant recipient to promote survival of the transplant or to treat transplant-related symptoms (e.g., cytokine release syndrome, neurotoxicity). Examples of other transplant-related therapies include, but are not limited to, administration of antibodies, antigen-targeting ligands, non-immunosuppressive drugs, and other agents that stabilize or destabilize components of transplants that are critical to transplant activity or that directly activate or inhibit one or more transplant activity. These activities may include the ability to induce an immune response, recognize particular antigens, replicate, and/or induce repair of damaged tissues. Adjusting immunosuppressive therapy may be combined with adjusting, initiating, or discontinuing other transplant-related therapies.

The methods of the disclosure may classify the status of a transplant, e.g., an organ transplant. The status of the transplant can be used to inform the need to adjust monitoring of the transplant recipient. In general, changes in the predictive rejection classification over time may be informative with regard to determining a need to adjust monitoring of a transplant recipient. In some embodiments, classifying the status of a transplant, as described above, is informative with regard to determining a need to adjust monitoring of a transplant recipient.

Depending on the status of the transplant, monitoring of the transplant recipient may be adjusted accordingly. For example, monitoring may be adjusted by increasing or decreasing the frequency of monitoring, as appropriate. Monitoring may be adjusted by altering the means of monitoring, for example, by altering the metric that is used to monitor the transplant recipient.

### Example System for Classifying the Status of a Transplant

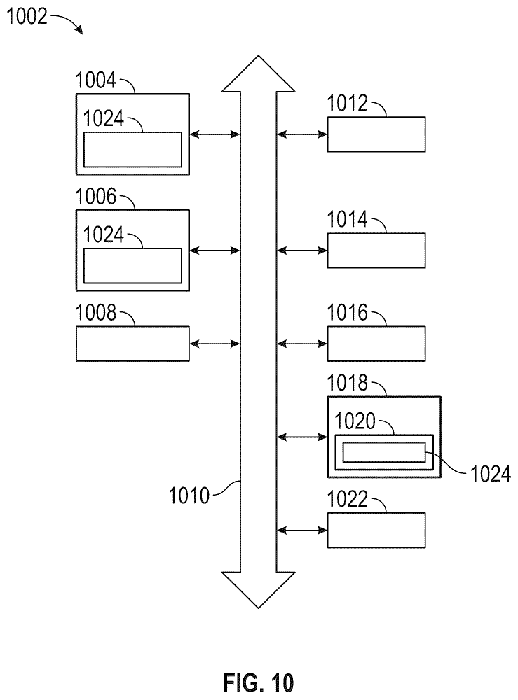

The system and methods discussed herein may be implemented by a device. FIG. 10 illustrates an example device that implements the disclosed system and methods, according to embodiments of the disclosure. The device 1002 may be a portable electronic device, such as a cellular phone, a tablet computer, a laptop computer, or a wearable device. The device 1002 can include a processor 1004 (e.g., a central processing unit (CPU), a graphics processing unit (GPU), or both), a main memory 1006 (e.g., read-only memory (ROM), flash memory, dynamic random access memory (DRAM) such as synchronous DRAM (SDRAM) or Rambus DRAM (RDRAM), etc.), and a static memory 1008 (e.g., flash memory, static random access memory (SRAM), etc.), which can communicate with each other via a bus 1010.

The device 1002 may also include a display 1012, an input/output device 1014 (e.g., a touch screen), a transceiver 1016, and storage 1018. Storage 1018 includes a machine-readable medium 1020 on which is stored one or more sets of instructions 1024 (e.g., software) embodying any of the methods or functions described herein. The software may also reside, completely or at least partially, within the main memory 1006 and/or within the processor 1004 during execution thereof by the device 1002. The one or more sets of instructions 1024 (e.g., software) may further be transmitted or received over a network via a network interface device 1022.

While the machine-readable medium 1020 is shown in an embodiment to be a single medium, the term “machine-readable medium” should be taken to include a single medium or multiple media (e.g., a centralized or distributed database and/or associated caches and servers) that store the one or more sets of instructions. The term “machine-readable medium” shall also be taken to include any medium that is capable of storing, encoding, or carrying a set of instructions for execution by the device and that causes the device to perform any one or more of the methods of the present invention. The term “machine-readable medium” shall accordingly be taken to include, but not be limited to, solid-state memories, optical and magnetic media, and carrier wave signals.

The system and methods described herein and the corresponding data can be stored in storage 1018, main memory 1006, static memory 1008, or a combination thereof. The display 1012 may be used to present a user interface to a physician who is treating transplant recipients or a medical expert, and the input/output device 1014 may be used to receive input (e.g., clicking on a graphic representative of a microblog) from the treating physician or medical expert. The transceiver 1016 may be configured to communicate with a network, for example.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A method for classifying a status of a transplant, the method comprising:
receiving expression levels of a plurality of genes from a biological sample of a transplant recipient;
receiving a plurality of sets of weights for the plurality of genes;
generating one or more probability rejection scores of one or more rejection labels based on the plurality of sets of weights and the expression levels; and
assigning a predictive rejection classification of the biological sample of the transplant recipient based on the one or more probability rejection scores, wherein the predictive rejection classification classifies the status of the transplant.

2. The method of claim 1, wherein at least one of the plurality of genes is associated with one or more of: immune cell activation, organ-specific defense against pathogens, regulation of tissue and cellular processes, or transcription regulation.

3. The method of claim 1, wherein the predictive rejection classification classifies the status of the transplant as experiencing antibody-mediated rejection (ABMR), T-cell mediated rejection (TCMR), mixed ABMR+TCMR, or no rejection.

4. The method of claim 1, wherein generating one or more probability rejection scores of one or more rejection labels comprises:
for each rejection label of a plurality of rejection labels, generating a probability rejection score based on the plurality of sets of weights and the expression levels.

5. The method of claim 1, wherein each set of weights comprises a weight for a corresponding rejection label.

6. The method of claim 1, wherein the plurality of sets of weights for the plurality of genes is from a machine-learning model trained to:
receive a discovery dataset from biological samples of a discovery cohort of transplant recipients, wherein the discovery dataset comprises gene expression levels of a plurality of genes and rejection classifications;
analyze the gene expression levels of the discovery dataset for associations with the rejection classifications in the discovery dataset;
identify a subset of genes from the plurality of genes of the discovery dataset; and
generate the plurality of sets of weights for the subset of genes based on the associations between the gene expression levels of the discovery dataset and the rejection classifications of the discovery dataset, wherein each set of weights is associated with one gene of the subset of genes.

7. The method of claim 6, wherein at least some of the rejection classifications of the discovery dataset comprise antibody-mediated rejection (ABMR), T-cell mediated rejection (TCMR), mixed ABMR+TCMR rejection, or no rejection.

8. The method of claim 6, wherein the machine-learning model was validated by:
acquiring a validation dataset from biological samples of a validation cohort of transplant recipients, wherein the validation dataset comprises gene expression levels for a plurality of genes and rejection classifications;
determining one or more computer-determined predictive rejection classifications from the validation dataset;
comparing one or more of the rejection classifications in the validation dataset and the one or more computer-determined predictive rejection classifications; and
determining a diagnosis accuracy based on the comparison, wherein the diagnosis accuracy is greater than a predetermined value.

9. The method of claim 1, wherein at least one gene of the plurality of genes comprises a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1.

10. The method of claim 1, wherein the transplant recipient received a transplant comprising one or more of: a kidney transplant, a heart transplant, a lung transplant, a pancreas transplant, a liver transplant, an intestinal transplant, or a vascularized composite allograft transplant.

11. The method of claim 1, wherein the transplant recipient received a transplant that is an allograft or a xenograft.

12. The method of claim 1, wherein the biological sample is an organ tissue sample.

13. A kit for classifying the status of a transplant, the kit comprising:
one or more probesets specific for one or more genes identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1, reagents, controls, and instructions for use.

14. The kit of claim 13, wherein the kit further comprises instructions for:
receiving expression levels of a plurality of genes from a biological sample of a transplant recipient;
receiving a plurality of sets of weights for the plurality of genes;
generating one or more probability rejection scores of one or more rejection labels based on the plurality of sets of weights and the expression levels; and
assigning a predictive rejection classification of the biological sample of the transplant recipient based on the one or more probability rejection scores, wherein the predictive rejection classification classifies the status of the transplant.

15. The kit of claim 13, wherein the predictive rejection classification classifies the status of the transplant as experiencing antibody-mediated rejection (ABMR), T-cell mediated rejection (TCMR), mixed ABMR+TCMR, or no rejection.

16. The kit of claim 13, wherein generating one or more probability rejection scores of one or more rejection labels comprises:
for each rejection label of a plurality of rejection labels, generating a probability rejection score based on the plurality of sets of weights and the expression levels.

17. The kit of claim 13, wherein the transplant recipient received a transplant comprising one or more of: a kidney transplant, a heart transplant, a lung transplant, a pancreas transplant, a liver transplant, an intestinal transplant, or a vascularized composite allograft transplant.

18. A system for classifying a status of a transplant, the system comprising:
a scoring unit that:
receives expression levels of a plurality of genes from a biological sample of a transplant recipient;
receives a plurality of sets of weights for the plurality of genes;
generates one or more probability rejection scores of one or more rejection labels based on the plurality of sets of weights and the expression levels; and
assigns a predictive rejection classification of the biological sample of the transplant recipient based on the one or more probability rejection scores, wherein the predictive rejection classification classifies the status of the transplant.

19. The system of claim 18, wherein at least one of the plurality of genes is associated with one or more of: immune cell activation, organ-specific defense against pathogens, regulation of tissue and cellular processes, or transcription regulation.

20. The system of claim 18, wherein the predictive rejection classification classifies the status of the transplant as experiencing antibody-mediated rejection (ABMR), T-cell mediated rejection (TCMR), mixed ABMR+TCMR, or no rejection.

21. The system of claim 18, wherein generate one or more probability rejection scores of one or more rejection labels comprises:
for each rejection label of a plurality of rejection labels, generate a probability rejection score based on the plurality of sets of weights and the expression levels.

22. The system of claim 18, wherein each set of weights comprises a weight for a corresponding rejection label.

23. The system of claim 18, wherein the plurality of sets of weights for the plurality of genes is from a machine-learning model trained to:
receive a discovery dataset from biological samples of a discovery cohort of transplant recipients, wherein the discovery dataset comprises gene expression levels of a plurality of genes and rejection classifications;
analyze the gene expression levels of the discovery dataset for associations with the rejection classifications in the discovery dataset;
identify a subset of genes from the plurality of genes of the discovery dataset; and
generate the plurality of sets of weights for the subset of genes based on the associations between the gene expression levels of the discovery dataset and the rejection classifications of the discovery dataset, wherein each set of weights is associated with one gene of the subset of genes.

24. The system of claim 23, wherein at least some of the rejection classifications of the discovery dataset comprise antibody-mediated rejection (ABMR), T-cell mediated rejection (TCMR), mixed ABMR+TCMR rejection, or no rejection.

25. The system of claim 23, wherein the machine-learning model was validated by:
acquiring a validation dataset from biological samples of a validation cohort of transplant recipients, wherein the validation dataset comprises gene expression levels for a plurality of genes and rejection classifications;
determining one or more computer-determined predictive rejection classifications from the validation dataset;
comparing one or more of the rejection classifications in the validation dataset and the one or more computer-determined predictive rejection classifications; and
determining a diagnosis accuracy based on the comparison, wherein the diagnosis accuracy is greater than a predetermined value.

26. The system of claim 18, wherein at least one gene of the plurality of genes comprises a gene identified from a group consisting of KIR_Inhibiting_Subgroup_1, IL7R, KLRK1, BK large T Ag, PLA1A, LGALS3, HLA-F, SMAD3, HLA-C, SH2D1B, CXCL11, GBP4, SFTPC, SOST, AGT, HSPA12B, NCAM1, NCR1, ITGA4, LCN2, HLA-DPB1, XCL1/2, BK VP1, COL4A1, ARG2, MCM6, CD59, CD69, SMARCA4, IL18, CMV UL83, SIGIRR, KIT, CD160, SERPINE1, TFRC, CCR7, HLA-B, CXCL8, AQP2, SOD2, SFTPB, HLA-DQA1, IFI6, HFE, MAPK12, GDF15, IFIT1, KLRF1, SERINC5, FOXP3, BCL2L1, FABP1, CCL21, LOX, ROBO4, MYBL1, AGR3, CXCR6, CXCL13, FCER1A, BTG2, CTLA4, CASP3, SPRY4, RAF1, MAPK13, IGF2R, RHOU, LYVE1, CD80, KAAG1, CCL18, EHD3, IL1RL1, CRIP2, TNFSF9, CDH5, CD8B, PRDM1, SIRPG, ABCA1, ADORA2A, RASSF9, JUN, COL4A4, TRAF4, PIN1, SOX7, CFB, CFH, SFTPD, THBS1, AIRE, RAMPS, IL1R2, GNG11, RAPGEF5, DEFB1, GNLY, PHEX, ENG, BMP7, RELA, COL1A1, PLAAT4, CD81, ICAM2, PLAT, CD40LG, NPHS2, IL33, CD58, TIPARP, TNC, PECAM1, C5, EGFR, CD2, BMP2, CTNNB1, MYB, CRHBP, MT2A, EEF1A1, BCL2, SLC19A3, VMP1, PSEN1, MAPK3, TFF3, TNFSF4, CD55, PDPN, IL17RB, IGHG2, CXCL12, CD207, MICA, MMP9, EOMES, EPO, NOS3, KLF2, KLF4, SLC4A1, P2RX4, CCL3/L1, and HPRT1.

27. The system of claim 18, wherein the transplant recipient received a transplant comprising one or more of: a kidney transplant, a heart transplant, a lung transplant, a pancreas transplant, a liver transplant, an intestinal transplant, or a vascularized composite allograft transplant.
